(19) United States
(12) Patent Application Publication
(10) Pub. No.: US 2023/0140574 A1
(43) Pub. Date: May 4, 2023

(54) NUCLEIC ACID PURIFICATION FROM FIXED BIOLOGICAL SAMPLES

(71) Applicant: QIAGEN GmbH, Hilden (DE)

(72) Inventors: Dominic O'NEIL, Hilden (DE); Stefanie SCHROEER, Hilden (DE); Christian KUPFER, Hilden (DE)

(21) Appl. No.: 17/907,610

(22) PCT Filed: Mar. 31, 2021
(86) PCT No.: PCT/EP2021/058514

§ 371 Date: Sep. 28, 2022

(30) Foreign Application Priority Data
Mar. 31, 2020 (EP) 20167445.4

(51) Int. Cl. C12Q 1/6806; C12Q 1/6844; C12N 15/10

(52) U.S. Cl. CPC C12Q 1/6806; C12Q 1/6844; C12N 15/1006; C12Q 1/6869

(57) ABSTRACT

The present invention provides a method for lysing a fixed biological sample, wherein the fixed biological sample comprises crosslinks between nucleic acid molecules and protein molecules due to the fixation, said method comprising (a) lysing the fixed biological sample, wherein lysis involves digestion with a proteolytic enzyme; (b) heating the lysed sample to reverse crosslinks; (c) adding a proteolytic enzyme and performing a proteolytic digestion; optionally wherein one or more additional treatment steps are performed between step (b) and step (c). The provided nucleic acids are of high yield and quality and can be purified from the lysed sample.

### FIELD OF THE DISCLOSURE

The present invention pertains to methods for lysing a fixed biological sample and/or obtaining purified nucleic acids from a fixed biological sample.

### BACKGROUND

Fixation of biological samples allows long-term preservation and archiving of the samples. Fixation is regularly achieved by protein-precipitating or protein-crosslinking compounds such as acids, alcohols, ketones or other organic substances such as glutaraldehyde, formaldehyde and/or paraformaldehyde. Such fixatives are well-known in the art. Fixation with formaldehyde (e.g. used in the form of a 35 percent by weight aqueous solution referred to as “formalin”), which can be followed by embedding of the fixed material in paraffin (called “formalin-fixed, paraffin-embedded” (FFPE) material) is a widely used fixation technology. Formaldehyde-based fixation has the advantage that tissue structures are attained comparatively well in the fixation. Fixation involving cross-linking fixatives are also used for fixing and thus preserving liquid samples. A commonly used preservative that comprises ethanol and formaldehyde is SurePath®.

Fixed biological samples (e.g. FFPE samples) are a valuable resource for investigating diseases. However, such samples are primarily used for histopathology, and are poorly suited for nucleic acid extraction and analysis, due to extensive damage and crosslinking of DNA resulting from the exposure to formaldehyde and storage. The most critical quality issue with DNA extracted from fixed biological samples is the crosslinking of the nucleic acids to protein and to each other. This crosslinking makes the DNA inaccessible to enzymes during reactions such as PCR, leading to very poor performance, and potentially false results in tests. Consequently, the efficient release and purification of nucleic acids (DNA or RNA) fixed biological samples (solid or liquid) is difficult. For numerous investigations on a molecular level, in particular for clinical or diagnostic applications, however, analysis of the nucleic acids is of great importance.

Methods to extract nucleic acids such as DNA from such fixed biological samples must reverse the cross-links that were introduced due to the fixation while preventing any further damage to the nucleic acids. A standard method of de-crosslinking DNA is to treat the crude lysate with heat e.g. for 1 hour at 90° C. or 4 hours at 80° C. and such de-crosslinking steps have all been used to dissolve the cross-links present in the fixed biological samples. Numerous commercial kits and methods for the extraction of FFPE are available in the art. All kits use a combination of heat, enzymes, and chemical lysis in order to remove the tissue from paraffin, digest the tissue, and purify the DNA. Further methods for isolate/release nucleic acids from fixed samples have been described in WO 2007/068764, WO 2014/072366, WO2005/075642, WO 2001/46402 and US 2005/0014203. Most of the available kits and methods have to make tradeoffs in terms of total yield and/or quality of DNA that is extracted (i.e. a higher DNA yield is often accompanied by fragmentation, while largely intact, high molecular weight DNA is often not completely de-crosslinked and is of worse quality in PCR applications). Further Important indicators for assessing the quality of extracted nucleic acid are the performance in PCR and/or NGS applications.

It is an object to provide a method for lysing a fixed biological sample that effectively releases contained nucleic acids, such as DNA and/or RNA, in particular DNA. It is furthermore an object of the present invention to provide a method for purifying nucleic acids, such as DNA and/or RNA from a fixed biological sample. In embodiments, it is an object of the present invention to avoid a drawback from the prior art methods. In embodiments, the methods of the present invention provide an improvement in at least one criterion, e.g. yield, fragmentation and/or performance in a subsequent nucleic acid analysis method, such as PCR and/or next generation sequencing.

### SUMMARY OF THE INVENTION

The present invention provides an improved method for lysing a fixed biological sample, wherein the fixed biological sample comprises crosslinks between nucleic acid molecules and protein molecules. These crosslinks are present because of the used fixation (e.g. formaldehyde and/or paraformaldehyde based fixation). It was found that the lysis and digestion of fixed biological samples can be significantly improved by a step-wise sequential lysis process, wherein the fixed biological sample is digested with a proteolytic enzyme, followed by heating to reverse crosslinks, followed by adding a proteolytic enzyme to perform an additional proteolytic digestion. The examples show that performing a second proteolytic digestion after performing a crosslink reversal step results in important and unexpected improvements.

According to a first aspect of the invention, a method is provided for lysing a fixed biological sample, wherein the fixed biological sample comprises crosslinks between nucleic acid molecules and protein molecules due to the fixation, the method comprising
(a) lysing the fixed biological sample, wherein lysis involves digestion with a proteolytic enzyme;
(b) heating the lysed sample to reverse crosslinks;
(c) adding a proteolytic enzyme and performing a proteolytic digestion;

optionally wherein one or more additional treatment steps are performed between steps (b) and (c).

As is demonstrated by the examples, the method according to the first aspect significantly improves the release of nucleic acids such as DNA compared to prior art methods. The fixed biological sample may be a solid biological sample (e.g. a fixed tissue sample) or a liquid biological sample (e.g. a fixed cell-containing liquid sample).

According to a second aspect of the invention, a method is provided for obtaining purified nucleic acids from a fixed biological sample, wherein the fixed biological sample comprises crosslinks between nucleic acid molecules and protein molecules due to the fixation, said method comprising lysing the fixed biological sample according to the lysis method of the first aspect, steps (a) to (c), wherein subsequent to step (c) of the method of the first aspect the method comprises
(d) purifying nucleic acids from the lysed sample.

As is disclosed herein, one or more additional treatment steps (e.g. at least one further enzymatic treatment step different from a proteolytic digestion step) may be optionally performed between steps (b) and (c) of the method according to the first aspect. The step-wise lysis/digestion procedure according to the first aspects improves the release of high quality nucleic acids (such as DNA) from the fixed biological sample, whereby the released nucleic acids (such as DNA) can then be purified from the digested sample in step (d) of the method according to the second aspect with high quality and/or high yield. Thereby, an improved method is provided for purifying nucleic acids, such as DNA, from a fixed biological sample.

A third aspect of the invention pertains to the use of a proteolytic enzyme, such as proteinase K, for performing a proteolytic digestion after lysing a fixed biological sample, wherein such prior lysis involves digestion of the fixed biological sample with a proteolytic enzyme and heating the lysed sample to reverse crosslinks, preferably in a method according to the first or second aspect of the present invention. As disclosed herein, the fixed biological sample comprises crosslinks between nucleic acid molecules and protein molecules due to the fixation. According to one embodiment, fixation involved the use of formaldehyde or paraformaldehyde.

A fourth aspect of the present invention pertains to the use of a glycosylase, such as a DNA glycosylase, preferably a uracil DNA glycosylase, for performing an enzymatic treatment, wherein the enzymatic treatment is completed in 30 min or less, 20 min or less, 15 min or less or 10 min or less. Such use may be performed in a method according to the first or second aspect of the present invention. In a preferred embodiment, the uracil glycosylase is a uracil-N-glycosylase.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### BRIEF DESCRIPTION OF THE FIGURES

FIG. 1: displays the DNA yield after extraction from different human FFPE tissues, including prostate (FIG. 1A), lung (FIG. 1B), kidney (FIG. 1C), spleen (FIG. 1D) and breast cancer (FIG. 1E). The DNA yield was determined using the QIAxpert (“UV-Vis”, dark shaded columns) and Qubit instruments (“dsDNA(Qubit)”, light shaded columns). Extraction using the high Tris lysis composition (“new GR—high Tris”) was compared to a low Tris lysis composition (“new GR—low Tris”) with (“+”) or without (“−”) an additional proteinase K digestion step (15 min, 65° C.).

FIG. 2: shows an electrophoresis gel of extracted DNA from human kidney and human breast cancer FFPE tissue samples. Extraction was performed using the high Tris lysis composition (“high Tris”) or the low Tris lysis composition (“low Tris”) with (“+”) or without (“−”) an additional proteinase K digestion step (15 min, 65° C.).

FIG. 3: Cq values of extracted DNA from breast cancer (top) or kidney FFPE tissue (bottom) measured by quantitative Real Time PCR using large amplicons (500 bp, right side) or short amplicons (66 bp, left side). Extraction using the high Tris lysis composition (“new GR—high Tris”) was compared to the low Tris lysis composition (“new GR—low Tris”) with or without an additional proteinase K digestion step (15 min, 65° C.). Results depicted in dark shaded columns correspond to same amount of DNA per reaction mixture and the light shaded columns correspond to same volume of diluted eluate per reaction mixture.

FIG. 4: shows the NGS results of extracted DNA from various human tissues. Shown is the number of reads per UMI (Unique Molecular Identifier, also referred to as Unique Molecular Index) of the extracted DNA. A value over 10 indicates that the same molecule was read more than 10 times, and is an indication of overamplification/not enough complexity in the starting material. Extraction was performed using high Tris lysis composition (“new GR—high Tris”) or the low Tris lysis composition (“new GR—low Tris”) with or without an additional proteinase K digestion step (“2^{nd} PK”).

FIG. 5: displays the DNA yield after extraction from different FFPE tissues (5A: human lung cancer tissue; 5B: human atrium tissue). The DNA yield was determined using the QIAxpert (“UV-Vis”, dark shaded columns) and Qubit instruments (“dsDNA(Qubit), light shaded columns”). Extraction using the high Tris lysis composition (“new GR—high Tris”) was compared to the low Tris lysis composition (“new GR—low Tris”) with (“15 min, 65° C. PK”) or without (“std”) an additional proteinase K digestion step. Moreover, an additional control was performed by performing the first proteinase K step over night at 56° C. (“o/n 56° C.”). As reference, extraction was also performed using the QIAamp® FFPE DNA kit (“QA”) as well as the Promega Maxwell RSC DNA FFPE kit and Maxwell RSC FFPE Plus DNA kit, wherein proteinase K digestion took place for 1 h at 70° C.

FIG. 6: shows an electrophoresis gel of extracted DNA from different FFPE tissues (6A: human lung cancer tissue; 6B: human atrium tissue). Extraction was performed using the high Tris lysis composition (“new GR—high Tris”) or the low Tris lysis composition (“new GR—low Tris”) with (“+65° C.”) or without (“std”) an additional proteinase K digestion step (15 min, 65° C.). Moreover, an additional control was performed by performing the first proteinase K step over night at 56° C. (“o/n”). As reference, extraction was also performed using the QIAamp® FFPE DNA kit (“QA FFPE”) as well as the Promega Maxwell RSC DNA FFPE kit and Maxwell RSC FFPE Plus DNA kit, wherein proteinase K digestion took place for 1 h at 70° C.

FIG. 7: Cq values of extracted DNA from different FFPE tissues (left: human lung cancer tissue; right: human atrium tissue) measured by quantitative Real Time PCR. In FIG. 7A short amplicons (66 bp) were used and in FIG. 7B large amplicons (500 bp). Extraction using the high Tris lysis composition (“new GR—high Tris”) was compared to the low Tris lysis composition (“new GR—low Tris”) with or without an additional proteinase K digestion step (15 min, 65° C.). Moreover, an additional control was performed by performing the first proteinase K step over night at 56° C. (“o/n 56° C.”). As reference, extraction was also performed using the QIAamp® FFPE DNA kit (“QA”) as well as the Promega Maxwell RSC DNA FFPE kit and Maxwell RSC FFPE Plus DNA kit, wherein proteinase K digestion took place for 1 h at 70° C. Results depicted in dark shaded columns correspond to same amount of DNA per reaction mixture and the light shaded columns correspond to same volume of diluted eluate per reaction mixture.

FIG. 8: shows the NGS results of extracted DNA from human atrium FFPE tissue. Shown is the number of reads per UMI (Unique Molecular Identifier also referred to as Unique Molecular Index) of the extracted DNA. A value over 10 indicates that the same molecule was read more than 10 times, and is an indication of overamplification/not enough complexity in the starting material. Extraction was performed using high Tris lysis composition (“new GR—high Tris”) or the low Tris lysis composition (“new GR—low Tris”) with or without an additional proteinase K digestion step (“2^{nd} K”) As reference, extraction was also performed using the QIAamp® FFPE DNA kit (“QA FFPE”) as well as the Promega Maxwell RSC DNA FFPE kit and Maxwell RSC FFPE Plus DNA kit, wherein proteinase K digestion took place for 1 h.

FIG. 9: shows an electrophoresis gel of extracted DNA from FFPE tissue samples. The left picture shows DNA extracted from FFPE rat heart, while the right picture shows DNA extracted from FFPE rat lung. The average size of the DNA extracted with the extraction method using the diluted lysis composition is much lower compared to the QIAamp® DNA FFPE Tissue reference protocol. (“GR std”=diluted lysis composition; “QA std”=reference lysis composition; L1-L3=DNA ladders as shown at the bottom).

FIG. 10: Cq values of extracted DNA measured by quantitative Real Time PCR using large amplicons (727 bp). DNA was extracted using a diluted lysis composition (“Fragmented”) and the reference protocol (“Standard”) from rat heart FFPE tissue.

FIG. 11: Cq values of extracted DNA measured by quantitative Real Time PCR using short amplicons (78 bp). DNA was extracted using a diluted lysis composition (“Fragmented”) and the reference protocol (“Standard”) from rat heart FFPE tissue.

FIG. 12: shows an electrophoresis gel of extracted DNA from FFPE tissue samples using high Tris lysis composition (“GR—high Tris”, “w/o”) or low Tris lysis composition (“GR—low Tris”, “GR std”) optionally containing additives, such as spermidine, spermine, DTT or glycine. (L1-L3=DNA ladders).

FIG. 13: Ct values of bisulfite DNA measured by quantitative Real Time PCR using short amplicons (110 bp). Different amounts of DNA were applied: 5 ng (dark shaded columns) or 10 ng (light shaded columns). Uracil nucleobases of the bisulfite DNA were removed in an enzymatic treatment step by providing a uracil-N-glycosylase (UNG). A higher Ct value thus indicates greater UNG activity. 5 or 10 ng DNA were measured for the lysis compositions of Example 4 in a 5 min UNG digestion step compared to a 60 min UNG digestion step (“GR FFPE std”). Further controls were performed without UNG (“w/o UNG”).

FIG. 14: Yield was determined by UV VIS and Qubit dsDNA BR measurement for a human kidney and a human breast sample (see Example 5). Displayed are average and standard deviation from 2 samples per condition.

FIG. 15: Performance in qPCR was determined by adding the same amount of volume adjusted to the actual elution volume to each reaction. A 66 bp and a 500 bp of the human 18S rRNA gene was amplified from eluates after extraction with either protocol option. Displayed are average and standard deviation from 2 samples per condition.

FIG. 16: Yield was determined by UV VIS and Qubit dsDNA BR measurement for a human heart sample (see Example 5). Displayed are average and standard deviation from 2 samples per condition.

FIG. 17: Performance in qPCR was determined by adding the same amount of volume adjusted to the actual elution volume to each reaction. A 66 bp and a 500 bp of the human 18S rRNA gene was amplified from eluates after extraction with either protocol option. Displayed are average and standard deviation from 2 samples per condition.

### DETAILED DESCRIPTION

The present invention provides an improved method for lysing a fixed biological sample, wherein the fixed biological sample comprises crosslinks between nucleic acid molecules and protein molecules due to the fixation. Furthermore, improved methods are described for obtaining purified nucleic acids such as DNA and/or RNA from a fixed biological sample.

The invention provides improvements in yield and/or quality of nucleic acids extracted from a fixed biological sample, such as FFPE tissue, enabling better analysis of the material, for instance using PCR and NGS sequencing methods. It was in particular found that an additional round of proteolytic digestion subsequent to a decrosslinking step (that is performed after a first proteolytic digestion step) provides important improvements.

In addition, the present disclosure provides methods that allow to control core parameters that are relevant for the analysis of fixed biological samples. In particular, it was found that by adjusting the lysis conditions in the first step it is possible to control the size of the released and this obtained nucleic acids. This important finding allows to optimize the methods for either short or long amplicon PCR systems. Furthermore, adjusting the lysis conditions as disclosed herein also improves the performance of enzymatic steps during the extraction, thereby enabling e.g. the in-process use of Uracil-n-glycolysase to remove artifacts caused by formalin cross-linking.

The different aspects and embodiments of the invention disclosed herein therefore make important contributions to the art.

### THE METHOD ACCORDING TO THE FIRST ASPECT

According to a first aspect of the invention, a method is provided for lysing a fixed biological sample, wherein the fixed biological sample comprises crosslinks between nucleic acid molecules and protein molecules due to the fixation, the method comprising
(a) lysing the fixed biological sample, wherein lysis involves digestion with a proteolytic enzyme;
(b) heating the lysed sample to reverse crosslinks;
(c) adding a proteolytic enzyme and performing a proteolytic digestion;

optionally wherein one or more additional treatment steps are performed between steps (b) and (c).

The individual steps and preferred embodiments will now be described in detail.

### Step (a)

The method according to the first aspect comprises in step (a) lysing the fixed biological sample, wherein lysis involves digestion with a proteolytic enzyme. During lysis step (a), the fixed biological sample is degraded such that nucleic acids are released.

Step (a) can be performed using lysis and proteolytic digestion conditions known in the art. Assisting the lysis of the fixed biological sample by digestion with a proteolytic enzyme is highly advantageous, as it allows degrading protein and peptide crosslinked to the nucleic acid. As disclosed herein and known in the art, such cross-links in the fixed biological sample are induced due to the used fixation, e.g. when using a crosslinking fixative. For instance, using a crosslinking fixative such as an aldehyde containing fixative, e.g. formaldehyde, leads to crosslinking between protein and nucleic acid, as well as between nucleic acids. By performing lysis step (a), nucleic acid-crosslinked protein can be degraded, as well as partially crosslinks between nucleic acids.

According to one embodiment, lysis step (a) comprises preparing a lysis mixture, wherein the lysis mixture comprises (i) the fixed biological sample, and (ii) a lysis composition comprising the proteolytic enzyme. Any contacting order of the fixed biological sample and the components of the lysis composition is encompassed by this embodiment for preparing the lysis mixture in step (a). E.g. the lysis composition may be prepared separate and the prepared lysis composition may then be contacted with the fixed biological sample or vice versa. Thus, the lysis composition can be first adjusted without affecting the fixed biological sample. It is furthermore within the scope of this embodiment that the lysis mixture is prepared by contacting the fixed biological sample in any order with the proteolytic enzyme and/or the further components of the lysis composition to prepare the lysis mixture.

The proteolytic enzyme supports the digestion of the fixed biological sample and improves the release of the comprised nucleic acids, such as DNA and/or RNA. In embodiments, the proteolytic enzyme is a protease, such as proteinase K. Proteases may either qualify as endopeptidases, which cleave peptide bonds within the protein, and/or are exopeptidases, which cleave off amino acids from the ends of a protein chain. Typically, proteases are further categorized by mechanism, such as serine proteases (e.g., chymotrypsin, trypsin, elastase, subtilisin, and proteinase K); cysteine (thiol) proteases (e.g., bromelain, papain, cathepsins, parasitic proteases, and bacterial virulence factors); aspartic proteases (e.g., pepsin, cathepsins, rerun, fungal and viral proteases); and metalloproteases (e.g., thermolysin). Such proteases may be used in the context of the present invention. In a core embodiment, the proteolytic enzyme used in step (a) is a serine protease. According to one embodiment, the proteolytic enzyme used in step (a) is a protease of the subtilisin family. A particularly suitable and preferred serine protease used in step (a) is proteinase K. Proteinase K is commonly used in the art for the digestion of fixed biological samples that comprise crosslinks because of the applied fixation. Proteinase K advantageously remains active even at higher temperatures and in the presence of a detergent, denaturing agents such as urea and chaotropic agents and salt. Proteinase K was also used in the present examples. All disclosures described herein for a proteolytic enzyme or protease in general specifically apply and particularly refer to the preferred embodiment proteinase K. As is apparent from the listed examples, the proteolytic enzyme may be a heat-stable protease. This allows to support the lysis and digestion in step (a) by heating the lysis mixture as is also described below. Examples of suitable heat-stable proteases are proteinase K, trypsin, chymotrypsin, papain, pepsin, pronase and endoproteinase Lys-C. The heat-stable protease may be inactivated at or above an inactivation temperature, which may lie in a range of 85° C.-120° C. Suitable inactivation temperatures for different proteases are described in the art for different proteases.

According to one embodiment, step (a) comprises heating to assist the digestion by the proteolytic enzyme. The prepared lysis mixture may thus be heated to a suitable elevated temperature that supports the lysis/digestion of the fixed biological sample. The lysis/digestion temperature is chosen such that the proteolytic enzyme is active. In embodiments, heating in step (a) is performed at a temperature in the range of 35-75° C., such as 40-70° C. or 45-65° C. As is known in the art, heating the lysis may enhance the activity of the proteolytic enzyme, thereby allowing for a fast and efficient sample lysis and digestion in step (a). According to one embodiment, digestion with a proteolytic enzyme in step (a) comprises heating to a temperature of at least 30° C., in particular at least 35° C., at least 40° C., at least 45° C., preferably at least 50° C.

According to one embodiment, the lysis mixture is incubated in step (a) for at least 15 min, such as at least 20 min, at least 25 or at least 30 min. In embodiments, the lysis mixture is incubated in step (a) for at least 45 min or at least 50 min. Advantageously, step (a) may be performed within a short timeframe. According to a one embodiment, step (a) is completed in 120 min or less. Step (a) may be completed in 100 min or less, 90 min or less or 70 min or less, such as about 60 min. Incubation may occur at an elevated temperature as described above. Furthermore, incubation, preferably assisted by heating as disclosed herein, may be supported by agitation. Therefore, the lysis mixture may be agitated during incubation in step (a). According to one embodiment, lysis and digestion with a proteolytic enzyme in step (a) comprises agitating and heating the lysis mixture at a temperature in the range of 35-75° C., such as 40-70° C. or 45-65° C. for 15 to 120 min, such as 20 to 100 min, 30 to 90 min or 45 to 75 min. Agitation may be performed by any method, such as shaking, rolling, inverting, etc.

The person skilled in the art can chose a suitable concentration of the proteolytic enzyme in the lysis composition and lysis mixture and suitable concentrations are known in the art. According to one embodiment, the proteolytic enzyme of step (a) is present in the lysis mixture and/or lysis composition at a concentration of at least 0.5 mg/mL, such as at least 1 mg/mL, at least 1.5 mg/mL or at least 2 mg/mL. Preferably, the concentration is at least 2.5 mg/mL or at least 3 mg/mL. According to one embodiment, the proteolytic enzyme used in step (a) is a serine protease, such as proteinase K, present in the lysis mixture and/or lysis composition at a concentration selected from 1-10 mg/mL, such as 1.5-7.5 mg/mL, 2-7 mg/mL, 3-6 mg/mL or 3.5-5 mg/mL. Such concentrations may be used when the fixed biological sample is a solid fixed biological sample, such as a fixed tissue sample. Such concentrations may also be used in the lysis mixture when processing fixed liquid biological samples. The lysis composition is in such case adapted to take into account any dilution effects because of the fixed liquid biological sample. For example, a dilution of the proteolytic enzyme by the liquid fixed biological sample may be compensated to providing a higher concentration of proteolytic enzyme in the lysis composition. Suitable concentrations can be readily calculated by the skilled person considering the suitable concentrations of proteolytic enzyme in the lysis mixture of step (a).

As disclosed above, step (a) comprises in a core embodiment preparing a lysis mixture, wherein the lysis mixture comprises (i) the fixed biological sample, and (ii) a lysis composition comprising the proteolytic enzyme.

In embodiments, the lysis mixture comprises the components of the lysis composition in the same or similar concentration as the lysis composition (e.g. allowing a deviation of up to 50%, such as up to 40%, up to 30%, or up to 20% or up to 10%). This is in particular when providing a fixed biological sample that is a solid sample, e.g. a fixed tissue sample. For instance, a solid fixed biological sample does not result in dilution of the compounds present in the lysis composition, i.e. the concentration of the compounds present in the lysis composition is the same or about the same as in the lysis mixture (with the solid fixed biological sample). In case the sample is a liquid fixed biological sample, the liquid sample dilutes the compounds of the lysis composition, so that typically a higher concentration of compounds is used in the lysis composition to take into account such dilution effect. For instance, a higher concentration of compounds can be provided in the lysis composition and/or a higher volume ratio of lysis composition can be provided in order to establish suitable conditions for lysis in the lysis mixture of step (a).

According to a preferred embodiment, the lysis composition in step (a) has a pH in the range of 6.0 to 9.5, preferably 6.5 to 9.0 or 7.0 to 9.0. An according pH range has been found to be particularly suitable for lysis step (a) according to the method of the present disclosure as is also demonstrated in the examples. In embodiments the pH is in a range of such as 7.0-8.0. In further embodiments, the pH is in the range of 8.0 to 9.0, such as 8.2 to 8.8. In particular, applying such alkaline pH in the lysis composition and thus in the lysis mixture led to less fragmentation as compared to more acidic conditions.

According to a preferred embodiment, the lysis composition in step (a) further comprises one or more, preferably all of the following compounds:
(i) a salt;
(ii) a detergent;
(iii) a buffering agent.

In a particular embodiment, the lysis composition comprises a salt, a detergent and a buffering agent. Optionally, the lysis composition further comprises a chelating agent, such as EDTA. The lysis composition has been found to be suitable for the method of the present disclosure, as is demonstrated in the examples. Individual embodiments of the compounds of the lysis composition are disclosed below.

According to a preferred embodiment, the lysis composition comprises at least one reactive compound which may act as a formaldehyde scavenger. A reactive compound is in particular capable of reacting with the fixative and/or reacting with crosslinks induced by the fixative. Preferably, one or two reactive compounds are comprised in the lysis composition of step (a). However, a reactive compound can also be added separately to the lysis mixture, e.g. in form of a solution or solid comprising the reactive compound.

According to a preferred embodiment, the reactive compound reacts with the fixative or a chemical moiety released in heating step (b) and/or with a crosslink induced by the fixative, such as a crosslink induced by an aldehyde-containing fixative, such as formaldehyde. For the biological sample may be fixed with a fixative, such as an aldehyde-containing fixative, e.g. formaldehyde or a formaldehyde derivative, which induces crosslinks between nucleic acid molecules and protein molecules or in-between protein or nucleic acid molecules. The reactive compound of the present invention can advantageously react with the fixative, e.g. formaldehyde. The fixative or chemical moiety derived therefrom that reacts with the reactive compound may be released during the heating step (b). The reactive compound reacts with the released fixative which has the advantage that the released fixative is removed from the equilibrium, which greatly favors de-crosslinking (see Kawashima et al., 2014, Clinical Proteomics, 2014, Vol. 11(4), “Efficient extraction of proteins from formalin-fixed paraffin-embedded tissues requires higher concentration of tris(hydroxymethyl)aminomethane”). Thus, the reactive compound functions as a scavenger, scavenging the fixative or chemical moiety derived therefrom that is released in decrosslinking step (b). Alternatively or additionally, the reactive compound may reacts with a crosslink that is induced by the fixative. In particular, the reactive compound may directly break down fixative-induced crosslinks comprised in the fixed biological sample, preferably in step (a) and/or step (b). Reaction of the reactive compound with crosslinks induced by the fixative preferably takes place during step (b) of the method of the present disclosure. Furthermore, the reactive compound can react with a fixative-induced crosslink that has formed throughout the reversible crosslinking reaction. For instance, an aldehyde-containing fixative, such as formaldehyde, can lead to a formation of an aminal group between two biological molecules, e.g. protein and DNA. The aminal group can reversibly form an imine group under release of a protonated amine group of one of the biological molecules. The imine group can then react with a reactive compound according to the present disclosure and thereby react with a crosslink induced by the fixative. Further conversion can then lead to imine base formation at the reactive compound under release of the second biomolecule. Hence, the crosslink induced by the fixative is reversed and the fixative is bound to the reactive compound. Advantageously the biomolecules, in particular nucleic acid, is released.

According to a preferred embodiment, the reactive compound comprises a nucleophilic group, preferably an amine group. The reactive compound may be selected from a nucleophilic agent as described in WO 2007/068764 A1. A reactive compound comprising a nucleophilic group was found particularly suitable to react with the fixative or chemical moiety released in heating step (b) and/or with a crosslink induced by the fixative, in particular a crosslink induced by an aldehyde-containing fixative, such as formaldehyde.

According to a preferred embodiment, the reactive compound comprises one or more primary amine groups, optionally one primary amine group and one or more hydroxyl groups, preferably three hydroxyl groups. The reactive compound may furthermore comprise two primary amine groups and optionally one secondary amine group. Such reactive compounds have been found to be particularly suitable for the present invention as demonstrated in the Examples. In particular, nucleic acid crosslinks were efficiently removed by the method of the present disclosure using such a reactive compound, leading to high quality which is particularly suitable for analysis methods, e.g. for PCR or NGS. Exemplary reactive compounds, which can be advantageously used are 2-amino-2-(hydroxymethyl)propane-1,3-diol or a derivative thereof or spermidine or a derivative thereof or a combination thereof. 2-amino-2-(hydroxymethyl)propane-1,3-diol may also be referred to as tris(hydroxymethyl)aminomethane or Tris.

According to a preferred embodiment, the reactive compound comprises at least two nucleophilic groups, preferably nucleophilic groups having different nucleophile strength. Such an embodiment has been found advantageous in the Examples, in particular resulting in nucleic acids that are of high quality which is particularly suitable for analysis methods, e.g. PCR or NGS. A first nucleophilic group of the reactive compound may be stronger than a second nucleophilic group. In an according reactive compound one or more types of nucleophilic groups can be present. For instance, one or two first nucleophilic groups and one, two or three second nucleophilic groups. According to one embodiment, the reactive compound comprises a first nucleophilic group which is a primary amine group and a second nucleophilic group which is a hydroxyl group or a secondary amine group. According to a particular embodiment, the reactive compound comprises a primary amine group and a hydroxyl group, preferably three hydroxyl groups. According to another embodiment, the reactive compound comprises a primary amine group, preferably two primary amine groups and a secondary amine group. According to an exemplary embodiment, the reactive compound is selected from 2-amino-2-(hydroxymethyl)propane-1,3-diol or a derivative thereof or spermidine or a derivative.

According to one embodiment, the reactive compound comprises a polyamine, preferably a natural polyamine, such as spermidine. Such a reactive compound has been found advantageous to modify the nucleic acid fragmentation, in particular by increasing the fragment size as demonstrated in the Examples.

A preferred reactive compound of the present disclosure is 2-amino-2-(hydroxymethyl)propane-1,3-diol, also referred to as Tris. Kawashima et al., 2014 describe that Tris acts as a formaldehyde scavenger by producing a Schiff base, cyclic hemiaminal and cyclic acetal adducts. Moreover, Tris may be directly involved in breaking down crosslinks, as a kind of transamination catalyst. Furthermore, Tris has the advantage that it can form a cyclic compound with the aldehyde-containing fixative, such as formaldehyde, and thus one molecule of Tris scavenges one molecule for aldehyde-containing fixative. As demonstrated in the Examples, 2-amino-2-(hydroxymethyl)propane-1,3-diol also allows to modify the nucleic acid fragmentation and also suitability for subsequent nucleic acid analysis methods. For instance, using 2-amino-2-(hydroxymethyl)propane-1,3-diol resulted in high PCR and NGS performance.

According to one embodiment, the lysis composition comprises more than one reactive compound. It may comprise two reactive compounds, wherein optionally the reactive compounds are selected from (i) a reactive compound comprising one or more primary amine groups, preferably one primary amine group, and one, two or three hydroxyl groups, preferably three hydroxyl groups, such as 2-amino-2-(hydroxymethyl)propane-1,3-diol, and (ii) a reactive compound comprising two primary amine groups and optionally one secondary amine group, such as spermidine.

According to one embodiment, the lysis mixture comprises a reactive compound in a concentration suitable for reacting with at least a portion of the fixative, in particular in heating step (b). According to one embodiment, the lysis mixture comprises one or more reactive compounds in a concentration suitable for reacting with at least a portion of the fixative, in particular in heating step (b). In embodiments, the portion of the fixative corresponds to at least 15% of the fixative present in the fixed biological sample, in particular at least 25%, at least 35%, at least 45%, at least 55%, at least 65%, at least 75% of the fixative present in the fixed biological sample.

According to a one embodiment, the reactive compound is present in the lysis composition and/or the lysis mixture in a concentration in a range of 1 mM to 500 mM or 5-500 mM in the lysis composition and optionally also in the lysis mixture.

According to one embodiment, the lysis mixture and/or the lysis composition comprises a reactive compound, which comprises two primary amine groups and preferably one secondary amine group, such as spermidine. Said reactive compound (e.g. spermidine) may be comprised in a concentration of at least 0.5 mM, such as at least 1 mM, at least 1.5 mM or at least 2 mM. The concentration may be selected from 0.25-25 mM, such as 0.5-20 mM, 1-15 mM, 1.25-10 mM or 1.5-7 mM, such as about 2.5 mM. Such a concentration has been found particularly advantageous for sample lysis, resulting in nucleic acids of high fragment size. Furthermore, by varying said concentration, the fragment size can be flexibly controlled. The lysis composition may comprise a buffering agent or a reactive compound which is also a buffering agent.

According to one embodiment, the lysis mixture and/or lysis composition comprise a reactive compound which comprises one or more primary amine groups, preferably one primary amine group, and one, two or three hydroxyl groups, preferably three hydroxyl groups, such as 2-amino-2-(hydroxymethyl)propane-1,3-diol. In embodiments, it is comprised in the lysis mixture and/or lysis composition in a concentration of at least 3 mM, such as at least 5 mM, at least 7 mM, or at least 10 mM. A suitable concentration may be selected from 3 mM-100 mM, in particular 5 mM-50 mM, 7 mM-30 mM, 9 mM-25 mM or preferably 10 mM-20 mM, such as 10 mM-15 mM. Such concentrations are advantageous for sample lysis, resulting in nucleic acids of small fragment size and high quality, in particularly for a nucleic acid analysis method involving amplification of small fragments, e.g. a short amplicon PCR, as disclosed herein. Furthermore, the high nucleic acid quality leads to enhanced NGS performance. Such applications are also disclosed in conjunction with the method according to the fifth aspect.

According to one embodiment, the lysis mixture and/or lysis composition comprise a reactive compound which comprises one or more primary amine groups, preferably one primary amine group, and one, two or three hydroxyl groups, preferably three hydroxyl groups, such as 2-amino-2-(hydroxymethyl)propane-1,3-diol. In embodiments, it is comprised in the lysis mixture and/or lysis composition in a concentration of at least 10 mM, such as at least 20 mM, at least 40 mM, at least 60 mM, at least 75 mM or at least 100 mM. The concentration may be selected from 10 mM-750 mM, such as 20 mM-500 mM, 30 mM-300 mM, 50 mM-250 mM or preferably 75 mM-200 mM, such as about 100 mM to 150 mM. This concentration has been found advantageous for sample lysis, resulting in nucleic acids having a high quality, particularly for a nucleic acid analysis method involving amplification of large and/or small fragments, in particular large amplicon PCR, as disclosed herein. It is referred to the method according to the sixth aspect. Furthermore, the high nucleic acid quality leads to enhanced NGS performance.

According to one embodiment, the reactive compound comprised in the lysis composition is additionally a buffering agent. In particular, both functions are advantageously fulfilled by such a compound, in particular buffering the lysis mixture and providing a reactive compound to react with the fixative or chemical moiety released in heating step (b) and/or with a crosslink induced by the fixative, in particular a crosslink induced by an aldehyde-containing fixative, such as formaldehyde. Such a compound may preferably comprise one or more primary amine groups, preferably one primary amine group, and one, two or three hydroxyl groups, preferably three hydroxyl groups, such as 2-amino-2-(hydroxymethyl)propane-1,3-diol. According to one embodiment, the lysis mixture and/or the lysis composition comprises a compound, which is a reactive compound and also buffering agent at a concentration selected from the range of 3-500 mM. Suitable concentration ranges were described above. As disclosed herein, the choice of the concentration of said reactive compound allows to control the fragment size of the nucleic acid molecules released during lysis.

Optionally, the lysis composition comprises two reactive compounds. In such an embodiment, preferably the first reactive compound is selected from a reactive compound, which comprises two primary amine groups and preferably one secondary amine group, such as spermidine, and the second reactive compound is selected from a reactive compound which comprises one or more primary amine groups, preferably one primary amine group, and one, two or three hydroxyl groups, preferably three hydroxyl groups, such as 2-amino-2-(hydroxymethyl)propane-1,3-diol. These embodiments are particularly advantageous for sample lysis and obtaining nucleic acids having an increased size or less fragmentation.

According to one embodiment, the lysis composition comprises the reactive compound in a concentration leading to a concentration in the lysis mixture suitable for reacting with at least a portion of the fixative, in particular in heating step (b). According to one embodiment, the above disclosed concentrations of the reactive compound in the lysis mixture correspond to the concentrations of the reactive compound in the lysis composition, in particular in case the fixed biological sample is a solid fixed biological sample, such as a fixed tissue sample. According to one embodiment, the lysis composition comprises the reactive compound in a concentration selected from the range of 0.25-500 mM, wherein the fixed biological sample is a solid fixed biological sample, in particular a fixed tissue sample. As demonstrated in the Examples, such a concentration of reactive compound, e.g. 2-amino-2-(hydroxymethyl)propane-1,3-diol and/or sperimidine, has been found advantageous for lysis of the fixed tissue samples, in particular by allowing to modify the nucleic acid size/fragmentation and obtaining nucleic acids of high quality, in particular suitable for a nucleic acid analysis method, e.g. PCR, NGS.

In case the fixed biological sample is a liquid fixed biological sample, the concentration of the reactive compound in the lysis composition in adapted to establish an above disclosed concentration in the lysis mixture. Alternatively or additionally, a higher volume ratio of lysis composition may be added to a liquid fixed biological sample in order to establish a concentration of the reactive compound in the lysis mixture as disclosed above. According to one embodiment, the lysis composition comprises the reactive compound in a concentration suitable to be in the lysis mixture present at a concentration selected from the range of 0.25-500 mM, wherein the fixed biological sample is a liquid fixed biological sample. According adjustment of the concentration of the reactive compound is well within the abilities of the skilled person.

According to one embodiment, the lysis composition and/or lysis mixture has a pH selected from the range of 6.0 to 9.5 and the lysis composition further comprises a reactive compound, in particular reacting with a fixative and/or reacting with a crosslink induced by the fixative, wherein the reactive compound comprises a nucleophilic group, in particular a primary amine group. Exemplary reactive agents are 2-amino-2-(hydroxymethyl)propane-1,3-diol or a derivative thereof and spermidine or a derivative thereof. According to one embodiment, the reactive compound is comprised in the lysis mixture and/or lysis composition in a concentration of 5-50 mM preferably 10-20 mM and the lysis composition has a pH in the range of 6.0 to 9.5, preferably 7.0 to 8.0. According to another embodiment, the reactive compound is comprised in the lysis mixture and/or lysis composition in a concentration of 60-250 mM, preferably 75-200 mM and the lysis composition has a pH in the range of 7.0 to 9.5, preferably 8.0 to 9.0. These embodiments are particularly advantageous, as they allows for improved release of a nucleic acid, e.g. DNA, from the fixed biological sample. By providing a neutral or slightly alkaline pH and such a reactive compound, the fixative of the fixed biological sample is advantageously removed by the reaction equilibrium by reacting with the reactive compound, allowing for releasing the nucleic acid with less fragmentation than at acidic conditions.

According to a preferred embodiment, the lysis composition comprises a salt. Salt supports the lysis. The salt is preferably provided to the lysis mixture by the lysis composition. The salt may preferably be a mono- or divalent salt. Preferably, the salt is a chaotropic or non-chaotropic salt. According to a preferred embodiment, the salt is a non-buffering salt. Also a mixture of salts can be used. A particular salt may be selected from an alkali metal salt, optionally an alkali metal halide. According to a preferred embodiment, the salt is a chloride salt, optionally selected from sodium chloride, potassium chloride, lithium chloride and cesium chloride, wherein preferably the salt is sodium chloride. Such salts have been found suitable as is demonstrated by the Examples.

The concentration of the salt in the lysis composition depends on the type of fixed biological sample from which the nucleic acid is released and can be flexibly adjusted by the skilled person. According to a preferred embodiment, the salt is present in the lysis composition and/or the lysis mixture in a concentration of at least 15 mM, in particular at least 30 mM, at least 50 mM, preferably at least 100 mM. Suitable concentration ranges of salt in the lysis composition and/or the lysis mixture may be selected from 15-500 mM, in particular 30-440 mM, 50-300 mM or preferably 100-250 mM, such as about 150 mM. According to one embodiment, the salt is present in the lysis composition and/or the lysis mixture in a concentration of less than 500 mM, in particular less than 400 mM, less than 300 mM or preferably less than 250 mM, such as less than 200 mM. Such salt concentrations have been found advantageous as demonstrated in the Examples for sample lysis. For instance, by applying such a salt concentration the digestion using a DNA glycosylase, such as a uracil DNA glycosylase, preferably a uracil-N-glycosylase, is advantageously completed in 30 min or less, 20 min or less, 15 min or less or 10 min or less. As disclosed above, when providing a solid fixed biological sample, the salt concentration in the lysis mixture corresponds to or is about the salt concentration in the lysis composition, while a liquid fixed biological sample dilutes the concentration such that it is lower in the lysis mixture than in the lysis composition. Accordingly, for a liquid fixed biological sample, the salt concentration can be adapted to establish the above disclosed concentration in the lysis mixture, in particular by providing a higher salt concentration in the lysis composition and/or providing a higher volume ratio of the lysis composition.

According to a preferred embodiment, the lysis composition comprises a detergent. Detergents support the lysis of the sample and dissolve protein aggregates. The detergent is preferably provided to the lysis mixture by the lysis composition.

According to a preferred embodiment, the detergent is a ionic or non-ionic detergent. Exemplary detergents are known by the skilled person. According to one embodiment, the detergent is an ionic detergent, preferably an anionic detergent. This is in particular suitable when the fixed biological sample is a solid fixed biological sample, such as a fixed tissue sample. A detergent in particular can be a sulfate or sulfonate of a fatty alcohol, such as sodium dodecyl sulfate, sodium dodecyl sulfonate or dodecylbenzenesulfonic acid, preferably the detergent is sodium dodecyl sulfate (SDS). Also a mixture of detergents can be used.

According to a preferred embodiment, the detergent is present in the lysis composition and/or the lysis mixture in a concentration of at least 0.01%, at least 0.02%, preferably at least 0.03%. Suitable concentration ranges of detergent in the lysis composition and/or the lysis mixture may be selected from 0.01-3.0%, 0.02-2.75%, preferably 0.03-2.5% or 0.04% to 2.0%. In embodiments, the concentration is in a range of 0.03-1%. As disclosed above, when providing a solid fixed biological sample, the detergent concentration in the lysis mixture corresponds to the concentration in the lysis composition, while a liquid fixed biological sample dilutes the concentration such that it is lower in the lysis mixture than in the lysis composition. Accordingly, for a liquid fixed biological sample, the detergent concentration can be adapted to establish the above disclosed concentration in the lysis mixture, in particular by providing a higher detergent concentration in the lysis composition and/or providing a higher volume ratio of the lysis composition.

According to one embodiment, the lysis composition comprises a buffering agent. Preferably, the buffering agent is selected from the group consisting of 2-amino (hydroxymethyl)propane-1,3-diol (also referred to as Tris), MOPS, HEPES, phosphate and borate, preferably selected from Tris. The buffering agent is preferably provided to the lysis mixture by the lysis composition. The buffering agent advantageously facilitates maintenance of the pH. As disclosed above, providing a suitable pH is particularly advantageous for the method according to the present disclosure and suitable pH ranges were disclosed above. Also a mixture of buffering agents can be used.

According to a preferred embodiment, the reactive compound is a buffering agent or the lysis composition or the lysis mixture comprises a buffering agent, optionally wherein the buffering agent has a pKa value which is in the range of 5.0 to 10.5, optionally selected from 5.5 to 10.0, 6.0 to 10.0, 6.5 to 10.0, 7.0 to 9.8 or 7.2 to 9.8.

According to one embodiment, the buffering agent is additionally a reactive compound, which is capable of reacting with a fixative and/or reacting with a crosslink induced by the fixative, optionally, it comprises a nucleophilic group, such as a primary amine group. For instance, the buffering agent may be 2-amino-2-(hydroxymethyl)propane-1,3-diol or a derivative thereof. In such an embodiment, the concentration disclosed above for the reactive compound corresponds to the concentration of the buffering agent.

According to one embodiment, the lysis composition comprises a chelating agent. The chelating agent can advantageously prevent nucleases from degrading target nucleic acids such as DNA. According to a preferred embodiment, the lysis composition further comprises a chelating agent, optionally wherein the chelating agent is an aminopolycarboxylic acid, preferably ethylenedinitrilotetraacetic acid (EDTA). According to one embodiment, the chelating agent is suitable for chelating divalent cations. Suitable chelating agents include but are not limited to diethylenetriaminepentaacetic acid (DTPA), ethylenedinitrilotetraacetic acid (EDTA), ethylene glycol tetraacetic acid (EGTA) and N,N-bis(carboxymethyl)glycine (NTA). According to a preferred embodiment, EDTA is used. As used herein, the term “EDTA” indicates inter alia the EDTA portion of an EDTA compound such as, for example, K₂EDTA, K₃EDTA or Na₂EDTA. Using a chelating agent such as EDTA has the advantageous effect that nucleases such as DNases and RNases are inhibited.

The chelating agent may be employed in a concentration of 0.05 mM to 5 mM, such as 0.075 mM to 2 mM, 0.1 mM to 1.5 mMin the lysis mixture and/or the lysis composition. A suitable concentration of the chelating agent in the lysis composition may depends on the applied sample type (solid or liquid fixed biological sample) and can be readily found by the skilled person considering suitable concentrations disclosed above for the lysis mixture.

According to one embodiment, the lysis composition is prepared by adding a reactive compound, wherein the reactive compound is selected from:
a reactive compound comprising a primary amine group and at least one hydroxyl group, preferably three hydroxyl groups, in particular 2-amino-2-(hydroxymethyl)propane-1,3-diol, and/or
a reactive compound comprising at least one primary amine group, preferably two primary amine groups, and a secondary amine group, in particular spermidine.

This embodiment is advantageous for flexibly modifying the type and concentration of the reactive compound independently from further compounds of the lysis composition. As demonstrated in the Example, by adding at least one of said reactive compounds, the nucleic acid size/fragmentation can be modified, e.g. for optimizing the nucleic acids for a nucleic analysis method. For instance, fragment size of nucleic acids have been increased, rendering the nucleic acids highly suitable for amplification of large nucleic acids as disclosed here, e.g. large amplicon PCR.

According to one embodiment, the lysis composition is aqueous and comprises:
a reactive compound comprising a primary amine, preferably it is selected from 2-amino-2-(hydroxymethyl)propane-1,3-diol and spermidine or a combination thereof; and
the proteolytic enzyme which is a protease, preferably a serine protease, more preferably proteinase K;

optionally the lysis composition further comprises
a detergent, preferably an anionic detergent, more preferably the detergent is sodium dodecyl sulfate;
a salt, preferably a monovalent salt, more preferably the salt is sodium chloride;
optionally, a chelating agent, preferably an aminopolycarboxylic acid, more preferably EDTA.

According to one embodiment, the lysis composition is aqueous and comprises:

(i) a reactive compound comprising a primary amine, preferably 2-amino-2-(hydroxymethyl)propane-1,3-diol, wherein said reactive compound is comprised in the lysis mixture and/or lysis composition in a concentration of (aa) at least 3 mM, in particular at least 5 mM, at least 7 mM, at least 9 mM or preferably at least 10 mM, e.g. 3-100 mM, in particular 5-50 mM, 7-30 mM, 9-25 mM or preferably 10-20 mM; or (bb) at least 10 mM, in particular at least 20 mM, at least 40 mM, at least 60 mM or preferably at least 75 mM, e.g. 10-1000 mM, in particular 20-500 mM, 40-300 mM, 60-250 mM or preferably 75-200 mM, such as about 100 mM to 150 mM;

or

(ii) a reactive compound comprising two primary amine groups and preferably one secondary amine group, wherein said reactive compound is comprised in the lysis mixture and/or lysis composition in a concentration of at least 0.25 mM, in particular at least 0.5 mM, at least 1 mM, at least 1.25 mM or preferably at least 1.5 mM, e.g. 0.25-25 mM, in particular 0.5-12.5 mM, 1-10 mM, 1.25-7.5 mM or preferably 1.5-5 mM, such as about 2.5 mM;

and

(iii) a protease enzyme, optionally a serine protease, preferably proteinase K;
optionally wherein the lysis composition further comprises:

(iv) a detergent, preferably an anionic detergent, more preferably the detergent is sodium dodecyl sulfate;

(v) a salt, preferably a monovalent salt, more preferably the salt is sodium chloride; and/or, preferably and

(vi) a chelating agent, preferably an aminopolycarboxylic acid, more preferably EDTA.

The combination of features of this lysis composition has been found advantageous for sample lysis as demonstrated in the Examples and has been disclosed herein.

According to one embodiment, the lysis composition comprises a reactive compound which is 2-amino-2-(hydroxymethyl)propane-1,3-diol, wherein said reactive compound is comprised in the lysis mixture and/or lysis composition in a concentration of 5-50 mM preferably 10-20 mM; wherein the lysis composition has a pH in the range of 6.0 to 9.5, preferably 7.0 to 8.0. This embodiment has been found advantageous as demonstrated in the Examples. In particular, high quality nucleic acids were obtained using this lysis composition, which are suitable for a nucleic acid analysis method involving amplification of small nucleic acids. Accordingly, in one embodiment using the lysis composition of this embodiment, nucleic acids are analyzed by amplifying nucleic acids smaller than 500 nt, which may be referred to as short amplicon PCR. Details are also disclosed elsewhere herein and it is referred to the respective disclosure.

According to one embodiment, the lysis composition comprises a reactive compound which is 2-amino-2-(hydroxymethyl)propane-1,3-diol, wherein said reactive compound is comprised in the lysis mixture and/or lysis composition in a concentration of 60-250 mM, preferably 75-200 mM; optionally wherein the lysis composition has a pH in the range of 7.0 to 9.5, such as 8.0 to 9.0. Also this embodiment has been found highly advantageous as demonstrated in the Examples. In particular, high quality nucleic acids were obtained using this lysis composition, which are suitable for a nucleic acid analysis method involving amplification of small and/or large nucleic acid molecules. Details of short and long amplicon PCR are disclosed elsewhere.

According to one embodiment, the lysis composition comprises a reactive compound which is spermidine, wherein said reactive compound is comprised in the lysis mixture and/or lysis composition in a concentration of 0.5-12.5 mM preferably 1.5-5 mM; and wherein the lysis composition further comprises 2-amino-2-(hydroxymethyl)propane-1,3-diol which is comprised in the lysis mixture and/or lysis composition in a concentration of 5-50 mM preferably 10-20 mM. This embodiment has been found highly advantageous as demonstrated in the Examples. In particular, high quality nucleic acids were obtained using this lysis composition, which are large in size and show less fragmentation.

According to one embodiment, the lysis composition is prepared by combining a lysis solution and the proteolytic enzyme. Optionally, further compounds are added to prepare the lysis composition, in particular additional reactive compound, as disclosed herein. Moreover, preparing the lysis composition may further comprise adding water.

According to one embodiment, the proteolytic enzyme combined with the lysis solution in step (a) is provided by a solution comprising the proteolytic enzyme. Suitable concentrations are disclosed elsewhere. The proteolytic enzyme can also be comprised in the lysis solution.

As disclosed herein, the lysis composition may be prepared by combining a lysis solution and the proteolytic enzyme, and optionally water or a dilution buffer.

According to one embodiment, the lysis solution comprises a salt, a detergent and a buffering agent. Moreover, the lysis solution in particular comprises a reactive compound. Details of said compounds were already disclosed above and it is referred to the reactive disclosure. Suitable concentration ranges of the reactive compound, detergent and salt have been disclosed herein for the lysis mixture and lysis composition. The lysis solution comprises the reactive compound, detergent and salt at a concentration suitable to establish a concentration as disclosed herein for the lysis mixture and/or lysis composition.

According to a preferred embodiment, the lysis solution comprises a reactive compound. A reactive compound has been disclosed herein and it is referred thereto. According to a particular embodiment, the lysis solution comprises a reactive compound comprising one or more primary amine groups, optionally one primary amine group and one or more hydroxyl groups, preferably three hydroxyl groups. According to a particular embodiment, the lysis solution comprises 2-amino-2-(hydroxymethyl)propane-1,3-diol or a derivative thereof.

According to a particular embodiment, the reactive compound is present in the lysis solution in a concentration of at least 5 mM, in particular at least 10 mM, at least 20 mM or at least 30 mM. The lysis solution may comprise 5-500 mM reactive compound, in particular 10-250 mM, 20-100 mM or 30-75 mM, such as about 53 mM. A particularly suitable reactive compound having said concentration in the lysis solution is a reactive compound comprising one or more primary amine groups, optionally one primary amine group and one or more hydroxyl groups, preferably three hydroxyl groups, such as 2-amino-2-(hydroxymethyl)propane-1,3-diol or a derivative thereof. According to a preferred embodiment, the reactive compound is a buffering agent.

According to one embodiment, the reactive compound comprises two primary amine groups and preferably one secondary amine group, such as spermidine, optionally wherein the reactive compound is present in the lysis solution sufficient for establishing a concentration of at least 0.25 mM in the lysis composition and/or lysis mixture, in particular at least 0.5 mM, at least 1 mM, at least 1.25 mM or preferably at least 1.5 mM. According to one embodiment, the lysis solution comprises a reactive compound as disclosed herein, in particular a reactive compound which is also a buffering agent, optionally wherein the reactive compound comprises one or more primary amine groups, preferably one primary amine group, and one, two or three hydroxyl groups, preferably three hydroxyl groups, such as 2-amino-2-(hydroxymethyl)propane-1,3-diol, optionally wherein the reactive compound is present in the lysis solution in a concentration of at least 5 mM, in particular at least 10 mM, at least 20 mM or at least 30 mM. Alternatively, the lysis solution comprises a buffering agent in said concentrations, wherein the buffering agent is not a reactive compound. Further reactive compound comprising two primary amine groups and preferably one secondary amine group can be added to the lysis composition or are comprised in the lysis solution, optionally wherein this reactive compound is present in the lysis solution sufficient for establishing a concentration of at least 0.25 mM in the lysis composition and/or lysis mixture, in particular at least 0.5 mM, at least 1 mM, at least 1.25 mM or preferably at least 1.5 mM. A reactive compound comprising two primary amine groups and preferably one secondary amine group in the above disclosed concentrations has been found advantageous for lysing the fixed biological sample and obtain nucleic acids of large fragment size, as is demonstrated in the Examples.

According to a preferred embodiment, the lysis solution comprises a salt, in particular a salt as disclosed herein. In particular the salt may be a chloride salt, such as sodium chloride, potassium chloride, lithium chloride and cesium chloride, preferably sodium chloride. According to one embodiment, the salt is present in the lysis solution in a concentration of at least 50 mM, such as at least 75 mM, at least 100 mM, at least 200 mM, at least 300 mM or at least 500 mM. According to one embodiment, the lysis solution comprises 75-2000 mM salt, in particular 100-1500 mM, 200-1200 mM, 300-1000 mM or 400-800 mM, such as about 500 to 700 mM. Advantages associated with said salt have been disclosed above and it is here referred thereto.

According to a preferred embodiment, the lysis solution comprises a detergent, in particular a detergent as disclosed herein. In particular, the detergent may be selected from a non-ionic or ionic detergent, preferably an anionic. In particular the detergent may be an anionic detergent, such as sulfate or sulfonate of a fatty alcohol, such as sodium dodecyl sulfate, sodium dodecyl sulfonate or dodecylbenzenesulfonic acid, preferably sodium dodecyl sulfate. According to one embodiment, the detergent has a concentration of at least 0.01% in the lysis solution, in particular at least 0.03%, at least 0.05%, at least 0.07% or at least 0.1%. According to one embodiment, the lysis solution comprises 0.01-3% detergent, in particular 0.03-2.5%, 0.05-2%, 0.07-1% or at least 0.1-0.5. Advantages associated with said detergent have been disclosed above and it is here referred thereto.

According to a particular embodiment, the lysis solution comprises
a reactive in a concentration of at least 5 mM, in particular at least 10 mM, at least 20 mM or at least 30 mM, e.g. 5-500 mM, in particular 10-250 mM, 20-100 mM or 30-75 mM, such as about 53 mM, in particular a reactive compound comprising one or more primary amine groups, optionally one primary amine group and one or more hydroxyl groups, preferably three hydroxyl groups, such as 2-amino (hydroxymethyl)propane-1,3-diol or a derivative thereof;
a salt in a concentration of at least 50 mM, optionally at least 75 mM, at least 100 mM, at least 200 mM, at least 300 mM or at least 400 mM, e.g. 75-2000 mM, in particular 100-1500 mM, 200-1200 mM, 300-1000 mM or 400-800 mM, such as about 600 mM, optionally wherein the salt is a chloride salt, such as sodium chloride, potassium chloride, lithium chloride and cesium chloride, preferably sodium chloride; and
a detergent in a concentration of at least 0.01% in the lysis solution, in particular at least 0.03%, at least 0.05%, at least 0.07% or at least 0.1%, e.g. 0.01-4%, in particular 0.03-3%, 0.05-2%, 0.07-1% or at least 0.1-0.5%, such as about 0.2%, optionally an anionic detergent, such as sulfate or sulfonate of a fatty alcohol, such as sodium dodecyl sulfate, sodium dodecyl sulfonate or dodecylbenzenesulfonic acid, preferably sodium dodecyl sulfate.

Such an embodiment is advantageous for the lysis of the fixed biological sample as has been demonstrated in the examples, wherein an according lysis solution has been applied.

Optionally, the lysis solution further comprises a chelating agent. According to one embodiment, the lysis solution further comprises a chelating agent, in particular EDTA, wherein the chelating agent has a concentration of at least 0.25 mM in the lysis solution, in particular at least 0.5 mM, at least 1 mM or at least 1.5 mM. According to one embodiment, the chelating agent has a concentration of 0.25-25 mM in the lysis solution, in particular 0.5-15 mM, 1-10 mM or 1.5-5 mM, such as 2.7 mM. The chelating agent advantageously supports inactivation of nucleases, such as DNases, as disclosed herein.

### Step (b)

Step (b) comprises heating the lysed sample to reverse crosslinks.

Performing a heating step (b) is advantageous as it allows to reverse the crosslinks induced by the fixative, such as e.g. formaldehyde induced crosslinks. These crosslinks are usually present between proteins, proteins and nucleic acid, as well as between nucleic acids. The crosslink reversal reaction is temperature dependent and a high temperature leads to a quicker reversal reaction (see Kennedy-Darling et al., Anal. Chem., 2014, Vol. 86 (12), pp: 5678-5681, “Measuring the Formaldehyde Protein—DNA Cross-Link Reversal Rate”). A temperature of at least 80° C. is particularly suitable for reversing the crosslinks induced by numerous fixatives, such as e.g. formaldehyde. Moreover, it is described in the art that heating after a proteolytic digestion step can remove chemical modifications of nucleic acids, which remain after the proteolytic digestion of step (a). For instance, it is described that methylol groups of nucleobases which can be present after a proteolytic digestion step can be removed by elevating the temperature (see e.g. Masuda et al., Nucleic Acid Research, 1999, Vol. 27 (22), pp: 4436-4443; “Analysis of chemical modification of RNA from formalin-fixed samples and optimization of molecular biology applications for such samples”).

Heating step (b) can also be employed to inactivate the proteolytic enzyme that is present in the lysis mixture. Inactivating the proteolytic enzyme in heating step (b) may be advantageous in case it is desired to perform one or more enzymatic treatments different than a proteolytic enzyme digestion step between step (b) and step (c). In one embodiment, heating step (b) inactivates the proteolytic enzyme that is present in the lysis mixture. Suitable inactivation conditions such as minimum inactivation temperatures and incubation times are described in the art for different proteases and thus are readily available to the skilled person.

Incubation temperatures and incubation times suitable to reverse fixative induced crosslinks are also described in the art. According to one embodiment, step (b) comprises heating the lysed sample to a temperature of at least 80° C., such as at least 85° C. or at least 90° C. According to one embodiment, step (b) comprises heating the lysed sample for at least 15 min, at least 20 min or at least 25 min in order to reverse crosslinks. In a preferred embodiment, step (b) comprises heating the lysed sample for at least 30 min, at least 45 min or at least 50 min. According to one embodiment, step (b) comprises heating the lysed sample at a temperature suitable for reversing crosslinks induced by the fixative up to 120 min, optionally up to 100 min. In embodiments, step (b) comprises heating up to 80 min or up to 70 min.

According to a preferred embodiment, the lysed sample is heated at a temperature in a range of 80-120° C., such as 80° C. to 110° C. or 85-100° C., for 30-120 min, such as for 45-90 min or 50-70 min. Step (b) may thus comprise heating the lysed sample at a temperature in the range of 80-110° C., such as 85-100° C., for 30-120 min. Step (b) may furthermore comprise heating the lysed sample at a temperature in the range of 80-110° C., such as 85-100° C., for 45-90 min. Step (b) may furthermore comprise heating the lysed sample at a temperature in the range of 80-110° C., such as 85-100° C., for 50-70 min.

In embodiments, at least 60%, at least 70%, at least 80%, at least 85%, at least 90% or at least 95% of the fixative induced crosslinks initially present in the fixed biological sample are reversed upon completion of step (b).

As is disclosed herein, one or more additional treatment steps may optionally be performed between the steps, and are in preferred embodiments performed between step (b) and step (c).

### Step (c)

Step (c) comprises adding a proteolytic enzyme and performing a proteolytic digestion. Therefore, the method according to the first aspect comprises performing at least one additional proteolytic digestion step subsequent to decrosslinking step (b). Accordingly, the method comprises first lysing the fixed biological sample, wherein lysis involves digestion with a proteolytic enzyme (step (a)), followed by heating the lysed sample to reverse crosslinks (step (b)), followed by adding a proteolytic enzyme and performing a proteolytic digestion (step (c)). This sequential order of steps was found particularly effective in lysing the fixed biological sample to release high quality nucleic acids with good yield. As is disclosed herein, one or more additional treatment steps may optionally be performed between steps (b) and (c).

As is demonstrated by the examples, performing step (c) significantly improves the release of nucleic acids during the overall lysis procedure. The released nucleic acids such as DNA and (or RNA) are of high quality and following purification, particularly suitable for amplification reactions. Surprisingly, performing an additional proteolytic digestion in step (c) improved the nucleic acid yield, as is demonstrated based on DNA in the examples. Without wishing to be bound in theory, it is assumed that the cross-links generated in the fixed biological sample (e.g. due to formaldehyde or other aldehyde-based fixatives) can cause particularly persistent protein associations with the nucleic acids or steric effects that may shield the protein from the proteolytic digestion performed in step (a). After heating step (b) which reverses crosslinks, these associations are weakened sufficiently that the additional proteolytic digestion step performed in step (c) is able to efficiently remove them. As is shown in the examples, performing step (c) surprisingly increases the yield and furthermore allows to release and thus obtain nucleic acids, such as DNA, of longer average size. This improvement in quality is beneficial for subsequent analyses of the released nucleic acids, as it also leads to better PCR results, in particular with large amplicons (500 bp). The results of the examples indicate that a larger amount of previously inaccessible long DNA strands have been made accessible to the subsequent PCR reaction when performing the additional proteolytic digestion step (c) after de-crosslinking step (b). These improvements that are demonstrated in the examples with respect to DNA yield and accessibility also result in an improved performance of the released DNA in downstream analytic processes, such as amplification and sequencing. The efficient removal of crosslinks and proteins that is achieved by the method according of the first aspect due to the sequential performance of steps (a), (b) and (c) is highly advantageous and also results in an improved NGS performance.

Therefore, performing step (c) as is taught by the method according to the first aspect leads to important and surprising improvements regarding the quality and yield of the released nucleic acid, such as particularly DNA.

The proteolytic enzyme used in step (c) is preferably a protease, such as proteinase K. Suitable proteases that can be used in step (c) were already disclosed in conjunction with step (a) and it is referred to the respective disclosure which also applies here. According to one embodiment, step (c) comprises adding proteolytic enzyme suitable for degrading protein and/or peptide associated with the nucleic acid and/or degrading crosslinks induced by the fixative, in particular crosslinks between the nucleic acid and a protein or peptide. According to one embodiment, the added proteolytic enzyme is a protease. Suitable proteases have been disclosed herein for step (a), and the same proteases may be used in step (c). As described above, preferably a serine protease is added in step (c), in particular proteinase K.

The proteolytic digestion in step (c) of the method of the present disclosure is preferably performed at a temperature suitable for degrading proteins and peptides by the proteolytic enzyme. Preferably, step (c) comprises heating the lysed sample to assist the digestion by the proteolytic enzyme. In embodiments, heating in step (c) is performed at a temperature in the range of 35-75° C., such as 40-70° C. or 50-70° C. In embodiments, the temperature is in the range of 55-68° C., such as 60° C. or 65° C. As is known in the art, the heating temperature of the used heating device may also be set to a higher temperature, wherein the preferred incubation temperature is reached in the proteolytic reaction mixture during the heating process (ramping) thereby assisting the proteolytic enzyme digestion before the higher temperature is then finally reached in the proteolytic reaction mixture. Such higher temperature may then also inactivate the proteolytic enzyme because the proteolytic digestion is completed before this inactivation temperature is reached in the course of the heating process.

To allow an efficient proteolytic digestion in step (c), the lysed sample may be incubated in the presence of the proteolytic enzyme. Incubation is preferably performed for at least 5 min, such as at least 10 min or at least 15 min. As disclosed herein, incubation for the proteolytic digestion may occur at an elevated temperature between 35-75° C. The sample may be agitated during incubation. According to one embodiment, step (c) comprises heating at a temperature of at least in 35° C., such as at least 40° C., at least 45° C., at least 50° C. or at least 55° C. for at least 5 min. Agitation may be performed by any method, such as shaking, rolling, inverting, etc.

According to one embodiment, step (c) comprises heating the lysis composition to a temperature selected from the range of 45° C. to 75° C., such 50° C. to 75° C. or 55° C. to 70° C. for 5-60 min, such as 10-45 min, 10-30 min, or 10-25 min. According to one embodiment, step (c) is completed in 30 min or less, optionally 20 min or less. As is demonstrated by the examples, a short incubation time such as 15 min (e.g. 65° C.) is feasible in step (c) and allows for significant improvements of yield and quality of nucleic acids comprised in the fixed biological sample. Moreover, such short incubation time enable the rapid completion of step (c), overall supporting that the method of the first aspect is not only highly efficient but also fast.

According to one embodiment, the incubation for the proteolytic digestion in step (c) and thus the time period for performing step (c) is shorter than the incubation in step (a) and thus the time period for performing step (a). Furthermore, the temperature used in step (c) to assist the proteolytic digestion may be higher in step (c) than in step (a).

The amount of the proteolytic enzyme added and the concentration of the proteolytic enzyme in the proteolytic reaction mixture of step (c) can be determined and chosen by the person skilled in the art. In one embodiment, the concentration of the proteolytic enzyme in the proteolytic reaction mixture is at least 0.5 mg/mL, preferably at least 1 mg/mL. Suitable concentration ranges include but are not limited to 0.5-10 mg/mL, 0.75-7.5 mg/mL, 1-5 mg/mL, or 1 to 3 mg/ml. Such concentrations have been applied in the examples. According to one embodiment, the proteolytic enzyme is added in step (c) is in form of a solution. The solution may comprise the proteolytic enzyme in a concentration of 1 mg/mL up to the solubility limit, e.g. in a concentration of 5-40 mg/mL, 7.5-35 mg/mL or 10-30 mg/mL, such as 20 mg/mL.

The examples show that step (c) advantageously supports the lysis of the fixed biological sample. In particular, nucleic acids are obtained with a higher yield and greater quality, which are highly suitable for a nucleic acid analysis method, such as PCR or NGS as disclosed herein.

### Optional Additional Treatment Steps Between Step (b) and Step (c)

Optionally, one or more additional treatment steps may be performed between steps (b) and (c). The additional treatments steps can be performed to further improve the method, e.g. in order to remove unwanted molecules. Non-limiting examples are the removal of RNA in case DNA is the target nucleic acid of interest (e.g. by performing a RNase digestion) or the removal of DNA in case RNA is the target nucleic acid of interest (e.g. by performing a DNase digestion). Furthermore, a treatment with a lipase may be performed. This can be advantageous, e.g. in case a fatty biological sample is processed. Furthermore, a treatment step can be performed to remove artifacts that are present due to the fixing of the biological sample, such as e.g. uracil nucleobases. This allows to provide nucleic acids such as DNA particularly suitable for amplification and sequencing.

According to one embodiment, the method according to the first aspect comprises performing at least one enzymatic treatment step different from a proteolytic digestion step between step (b) and step (c). According to one embodiment, the at least one enzymatic treatment step involves the use of one or more of a glycosylase, a nuclease, a lipase or a combination of the foregoing.

According to one embodiment, the at least one enzymatic treatment step involves the use of a DNA glycosylase, such as a uracil DNA glycosylase. If such step is performed, preferably an uracil-N-glycosylase treatment is performed between step (b) and step (c). According to one embodiment, the method comprises performing an enzymatic treatment step by adding a glycosylase to the lysed sample and heating. Using a glycosylate, such as a uracil glycosylase has important advantages. Fixed biological samples, such as for instance formalin-fixed and paraffin-embedded (FFPE) tissue samples, can have non-reproducible sequence artifacts in the DNA. Especially, C:G>T:A base substitution has been reported as the predominant type of sequence artifact in DNA recovered from fixed biological samples. This is based on cytosine deamination to uracil and sequential PCR amplification, leading to a C:G>T:A base substitution (see e.g. Do et al., Oncotarget, 2012, Vol. 3 (5): pp. 546-558, “Dramatic reduction of sequence artifacts from DNA isolated from formalin-fixed cancer biopsies by treatment with uracil-DNA glycosylase”). This can be advantageously avoided by treating the lysed sample after step (b) and prior to step (c) with a DNA glycosylase, in particular a uracil-DNA glycosylase. The enzyme removes the false uracil leading to formation of an abasic site. This in turn can induce breakage of the strand and/or block the DNA polymerase in a subsequent polymerization step. As a result, a potential artifact (C:G>T:A base substitution) is eliminated. This allows to improve the quality of the released DNA for subsequent sequencing applications. According to one embodiment, the glycosylase treatment is performed at an elevated temperature assisting the glycosylase activity. Suitable are e.g. a temperature in the range of 45-55° C., such as 50° C. In embodiments, the glycosylase treatment step is completed in 30 min or less, 20 min or less, 15 min or less or 10 min or less, optionally wherein the glycosylase is a uracil-N-glycosylase.

According to one embodiment, when performing the enzymatic treatment step the sample comprises a salt concentration suitable for performing the enzymatic treatment step, in particular an enzymatic treatment step comprising a glycosylase, preferably a DNA glycosylase, more preferably a uracil DNA glycosylase, such as a uracil-N-glycosylase; and/or a nuclease, preferably a ribonuclease, more preferably a ribonuclease A.

According to one embodiment, the method comprises diluting the lysed and decrosslinked sample prior to performing the at least one enzymatic treatment step. The sample can be diluted, e.g. by adding water or other dilution solution. Dilution can be advantageous to adjust suitable conditions for performing at least one enzymatic treatment between step (b) and step (c). Dilution can be e.g. advantageous to establish a salt concentration suitable for performing the desired enzymatic treatment step. In one embodiment, the salt concentration in the enzymatic treatment mixture generated for performing the enzymatic treatment step is ≤500 mM, such ≤300 mM or ≤250 mM. In embodiments, the salt concentration is ≤200 mM, ≤150 mM or ≤100 mM. Adjusting a sufficiently low salt concentration, optionally achieved by dilution of the lysed and cross-linked sample, ensures that the enzymatic treatment is not negatively affected by the salt. The suitable salt concentration depends on the intended enzymatic treatment and can be determined by the skilled person. For instance, providing a low salt concentration in the enzymatic treatment mixture, e.g. of less than 150 mM or less than 100 mM, was found particularly suitable for the enzymatic treatment using a uracil DNA glycosylase. In particular, it was found that these low salt conditions allow to reduce the treatment time. For instance, the treatment time can be reduced to less than 30 min, such as less than 25 min, less than 20 min, preferably less than 15 min or less than 10 min. Suitable time periods include 2-25 min, 2-20 min, 3-15 min and 5-10 min. As is demonstrated by the examples, an uracil-N-glycosylase digestion could be completed within 5 min. This is advantageous, as the method can be performed faster allowing a higher sample throughput.

According to one embodiment, at least a nuclease treatment step is performed in-between step (b) and step (c), optionally wherein the nuclease is a ribonuclease, such as ribonuclease A. The use of a ribonuclease allows to degrade RNA, thereby allowing to provide released DNA, devoid of RNA contaminations.

According to one embodiment, at least one, preferably both of the following enzymatic treatment steps are performed:
adding a glycosylase to the sample, preferably a DNA glycosylase, more preferably a uracil DNA glycosylase; and/or
adding a nuclease to the sample, optionally wherein the nuclease is a ribonuclease, such as ribonuclease A.

### The Method According to the Second Aspect

According to a second aspect of the invention, a method is provided for obtaining purified nucleic acids from a fixed biological sample, comprising lysing the fixed biological sample according to the lysis method of the first aspect, steps (a) to (c), wherein subsequent to step
(c) of the method of the first aspect the method comprises
(d) purifying nucleic acids from the lysed sample.

As disclosed herein, according to certain embodiment the method according to the first aspect comprises performing one or more further treatment steps in-between steps (b) and (c), such as a nuclease digestion step and/or a treatment with a uracil-N-glycosylase.

The method according to the second aspect advantageously provides purified nucleic acids from a fixed biological sample, wherein the nucleic acids are of high yield and quality. The high quality and yield of purified nucleic acids is advantageous, as it improves the subsequent analyses of the isolated nucleic acids, such as the amplification and/or sequencing of the purified nucleic acids. Sequencing may be performed by next generation sequencing. As is demonstrated in the examples, the method according to the second aspect provides pure nucleic acids that are particularly suitable for next generation sequencing applications. As disclosed herein, the method can be adjusted to control the size of the purified nucleic acids. This can be done as disclosed herein by choice of the lysis buffer that is used in step (a). Thereby, the degree of fragmentation can be adjusted and controlled. This allows to provide nucleic acids that are either optimized for short or long PCR systems.

The nucleic acids may be DNA or RNA and in embodiments the nucleic acids are DNA molecules.

Features of step (a), (b) and (c), as well as the one or more optional treatment steps between step (b) and step (c) have been disclosed above in conjunction with the method according to the first aspect and it is referred to the respective disclosure which also applies to the method according to the second aspect.

In one embodiment, at least one intermediate treatment step is performed between step (c) and step (d). In another embodiment, no further treatment step is performed between step (c) and step (d).

### Step (d)

The method according to the second aspect comprises step (d) purifying nucleic acids from the lysed sample. In step (d), any suitable purification method can be used, as the lysis method according to the first aspect renders the nucleic acids accessible in the lysis mixture. Suitable techniques for purifying nucleic acids from a lysed sample are known in the art and therefore, do not need any detailed description. Commercially available purification kits may be used in step (d).

According to a preferred embodiment, step (d) comprises
binding nucleic acids comprised in the lysed sample to a solid phase,
optionally washing the nucleic acids bound to the solid phase; and
eluting the nucleic acids from the solid phase.

According to one embodiment step (d) comprises contacting the lysed sample comprising the released nucleic acids (obtained after step (c)) with a binding composition (e.g. a binding buffer) in order to establish binding conditions for binding the target nucleic acids to the solid phase. The resulting mixture that establishes suitable binding conditions is also referred to as binding mixture herein. According to one embodiment, the binding composition comprises a chaotropic agent such as a chaotropic salt. Purification methods employing a chaotropic agent/chaotropic salt in order to promote binding of target nucleic acids to a solid phase are well-known in the art and therefore, do not need to be described in detail. Chaotropic salts include but are not limited to salts comprising guanidinium, iodide, perchlorate and thiocyanate and the chaotropic salt may be selected from guanidinium hydrochloride, guanidinium thiocyanate (GTC), guanidinium isothiocyanate (GITC), sodium thiocyanate, sodium iodide, sodium perchlorate, sodium trichloroacetate, sodium trifluroacetate. The binding composition and/or the binding mixture may furthermore comprise urea and/or a non-chaotropic salt. The binding composition and/or the binding mixture may furthermore comprise a detergent (e.g. an ionic or non-ionic detergent); and optionally an aliphatic alcohol, such as an alkanol, preferably comprising 1-5 or 2-3 carbon atoms. Ethanol or isopropanol are commonly used as alcohol for nucleic acid purification to promote binding to a solid phase. The mixture of the nucleic acids and the binding composition can then be applied to a solid phase or a solid phase may be added to said mixture. The solid phase may have a silica surface. The solid phase may comprise an unmodified silicon containing surface to which the target nucleic acids bind. The term “silica surface” as used herein includes surfaces comprising or consisting of silicon dioxide and/or other silicon oxides, diatomaceous earth, glass, silica gel, zeolithe, bentonite, alkylsilica, aluminum silicate and borosilicate. Exemplary solid phases that can be used in conjunction with the present invention include, but are not limited to, solid phases comprising an unmodified silica surface, including but not limited to, silica particles, silica fibres, glass materials such as e.g. glass powder, glass fibres, glass particles or controlled pore glass, silicon dioxide, glass or silica in particulate form such as powder, beads or frits. According to the present disclosure, the use of a column based solid phase or the use of particles, in particular magnetic particles, is preferred. According to one embodiment, the solid phase is comprised in a column. The column preferably comprises the solid phase having an unmodified silicon containing surface that is used for nucleic acid binding, particularly for DNA binding.

Other nucleic acid purification technologies that can be used in step (d) involve binding of the target nucleic acid to a solid phase having an anion exchange surface. Again, the solid phase may be provided in a column or bead format. Such methods are known in the art and therefore, do not need to be described in detail.

The solid phase with the bound nucleic acids may be separated from the remaining sample and the bound nucleic acids may be washed. The nucleic acids may furthermore be eluted. Elution solutions are well known by the skilled person and do not need to be further defined here.

Other nucleic acid purification methods can also be used in step (d), such as e.g. precipitation based purification methods. Such methods are known in the art and may involve the use of alcohols.

The target nucleic acids obtained according to the method according to the second aspect of the present disclosure may then be further processed and e.g. used for a nucleic analysis method as disclosed herein. As disclosed herein, the method according to the second aspects which is based on the lysis method according to the first aspect provides improvements in yield and quality of target nucleic acids extracted from fixed biological samples such as FFPE tissue. As is shown by the examples, the provided purified nucleic acids are better suitable for subsequent PCR and NGS analysis compared to prior art methods.

The target nucleic acids may be DNA and/or RNA. In one embodiment, the target nucleic acids to be bound comprise or substantially consist of DNA. As disclosed herein, the method according to the second aspect provides

### Further Embodiments of the Method According to the First and Second Aspect

### The Nucleic Acids

The “nucleic acid” or “nucleic acids” may be DNA and/or RNA. Therefore, DNA and RNA may be released from the fixed biological sample in the method according to the first aspect, or purified in the method according to the second aspect. Furthermore, the nucleic acid may be DNA or RNA. As disclosed herein, predominantly DNA (or RNA) can be obtained by either performing a nuclease treatment (e.g. during the method according of the first aspect) in order to destroy the non-target nucleic acid and/or a target nucleic acid selective purification may be performed in step (d) of the method according to the second aspect. This allows to obtain the target nucleic acid (e.g. DNA) with no or a low amount of non-target (e.g. RNA) contamination.

According to a preferred embodiment, the nucleic acids comprise or substantially consist of DNA. As is demonstrated in the examples, DNA can be very efficiently released from the fixed biological sample and subsequently purified.

### The Fixed Biological Sample

The term “fixed biological sample” in particular refers to any biological material that has been preserved with a fixative. The fixed biological sample comprises crosslinks between nucleic acid molecules and protein molecules due to the fixation. The used fixative is a cross-linking fixative. Such fixed biological samples include but not limited to formaldehyde-fixed tissues or organs, tissue samples stored in liquid cytological preservation media, and fixed cell-containing samples, stored in liquid cytological preservation material (such as cervical or gynecological swabs or cell-containing body fluids).

In one embodiment, the cross-linking fixative used for fixing the biological sample is an aldehyde containing fixative, such as formaldehyde and/or paraformaldehyde. Cross-linking fixatives include but are not limited to aldehyde compounds (such as formaldehyde, paraformaldehyde, and glutaraldehyde), osmium tetroxide, potassium dichromate, chromic acid, and potassium permanganate. Also included in this embodiment are fixatives known to release cross-linking compounds, such as formaldehyde over time. Formaldehyde is a well-known cross-reactive molecule that fixes the biological sample by cross-linking e.g. amino groups by methylene bridges. According to one embodiment, the biological sample was fixed using formaldehyde and/or paraformaldehyde. As is known in the art, formaldehyde can be used as fixative for solid and liquid biological samples.

According to a preferred embodiment, the fixed biological sample is a solid fixed biological sample, in particular a fixed tissue sample. Exemplary solid fixed biological samples include, but are not limited to tissues, including but not limited to, liver, spleen, kidney, lung, intestine, thymus, colon, tonsil, testis, skin, brain, heart, muscle and pancreas tissue. The fixed biological sample may furthermore be a fixed cell containing sample, such as a cell-containing body fluid and samples derived therefrom, aspirates, cell culture, bacteria, microorganisms, viruses, plants, fungi, biopsies, bone marrow samples, swab samples, faeces, skin fragments and organisms.

According to one embodiment, the fixed biological sample is a fixed tissue sample, wherein the tissue sample is an animal tissue, preferably a mammalian tissue sample, in particular a human tissue sample. The tissue may be obtained from autopsy, biopsy or from surgery.

According to a preferred embodiment, the fixed biological sample is a liquid fixed biological sample. Exemplary liquid fixed biological samples include, but are not limited to, fixed body fluid samples such as blood, serum, plasma, urine, saliva, tears, sweat, feces, mucous, breast milk, bone marrow, and spino-cerebral fluid.

In one embodiment, the fixed biological sample is a biological sample in a liquid cytological preservative medium. The liquid cytological preservative medium comprises a cross-linking fixative, such as an aldehyde-containing fixative, such as formaldehyde. Although the liquid cytological preservative medium is useful for cytology purposes, it can inhibit the efficient isolation of nucleic acids from the fixed biological samples. Exemplary aldehyde-containing fixatives commonly used in liquid cytology preservative medium include, but is not limited to, formaldehyde, glyoxal, glutaraldehyde, glyceraldehyde, acrolein, or other aliphatic aldehydes. One commonly used liquid cytological preservative medium that comprises a fixative is SUREPATH®, which is one of the most commonly used preservative media in clinical settings (e.g. to preserve swabs). SUREPATH® medium has a nearly 37% formaldehyde content and also contains methanol, ethanol, and isopropanol. The high formaldehyde content makes it a useful fixative, but poses challenges for subsequently extracting the target nucleic acids such as DNA from such fixed biological samples and using them for subsequent analysis. The methods according to the present invention provide can be advantageously used for such difficult fixed samples. According to one embodiment, the liquid fixed biological sample is a biological sample in SUREPATH®.

In one embodiment, the fixed biological sample is a solid cell-containing biological sample. The fixed biological sample may be embedded in a non-reactive embedding substance such as paraffin. According to one embodiment, the fixed biological sample is embedded in an embedding material, such as paraffin. According to one embodiment, the fixed biological sample is a fixed tissue sample that has been fixed using a cross-linking fixative (such as formaldehyde), and embedded in an embedding material, preferably in paraffin (such as a FFPE sample). As disclosed in the art, embedding materials include, but are not limited to, paraffin, mineral oil, non-water soluble waxes, celloidin, polyethylene glycols, polyvinyl alcohol, agar, gelatin or other media.

According to a particular embodiment, the fixed biological sample is an FFPE sample.

When processing fixed samples that have been embedded in an embedding material such as paraffin, it is within the scope of the present disclosure to include a step to remove said embedding material prior to step (a). According to one embodiment, prior to contacting step (a) the fixed biological sample is processed in order to remove any embedding material (such as paraffin) from the fixed biological sample. The removal of the embedding material (such as paraffin) from the biological sample can take place by any method known in the art for deparaffinization of biological samples. E.g. this may be achieved by contacting the sample with a hydrophobic organic solvent, such as xylene, in order to dissolve out the embedding material such as paraffin. Suitable deparaffinization methods are e.g. described in WO 2012/085261, WO 2011/104027, WO 2011/157683 and WO 2007/068764, as well as in the GeneRead™ DNA FFPE handbook (QIAGEN, March 2014) and the QIAamp® DNA FFPE Tissue Handbook (QIAGEN, June 2012) using the QIAGEN supplementary protocol on “Purification of genomic DNA from FFPE tissue using the QIAamp® DNA FFPE Tissue Kit and Deparaffinization Solution”.

### Nucleic Acid Analysis

According to a preferred embodiment, the method according to the second aspect comprises (e) analyzing the purified nucleic acids.

The nucleic acid analysis method may be any chemical and/or biotechnological method that can be used to analyze nucleic acids e.g. in order to amplify, identify, detect and/or quantify a nucleic acid. Preferably, the nucleic acid analysis method comprises a detection reaction which allows to detect the presence, absence and/or quantity of nucleic acids comprised in the enriched nucleic acid. Preferably, said step (e) comprises the amplification of at least one target nucleic acid. Respective analytical methods are well-known in the prior art and are also commonly applied in the medical, diagnostic and/or prognostic field in order to analyze nucleic acids or a specific nucleic acid comprised in or suspected to be comprised in the purified nucleic acids.

According to a preferred embodiment, the purified nucleic acid obtained in step (d) is used in a nucleic acid analysis method that comprises amplification. Such a method preferably involves enzymatic amplification, such as a polymerase-based amplification. In a particular embodiment, a polymerase chain (PCR) reaction is performed to amplify the enriched nucleic acid.

According to a particular embodiment, the method further comprises amplifying the nucleic acids using a large amplicon PCR and/or a short amplicon PCR. According to one embodiment, the large amplicon PCR is for nucleic acid molecules having a size of at least 500 bp. The short amplicon PCR is for nucleic acid molecules having a size of less than 500 bp, such as preferably 300 bp, 200 bp or 150. In embodiments, the short amplicon PCR is less than 100 bp.

In embodiments, a lysis composition comprising at least 10 mM, in particular at least 20 mM, at least 40 mM, at least 60 mM or preferably at least 75 mM reactive compound, optionally a reactive compound comprising a primary amine, preferably 2-amino-2-(hydroxymethyl)propane-1,3-diol for a large amplicon PCR. As disclosed herein, may be particularly advantageous to provide a lysis composition comprising at least 3 mM, in particular at least 5 mM, at least 7 mM, at least 9 mM or preferably at least 10 mM reactive compound, optionally a reactive compound comprising a primary amine, preferably 2-amino-2-(hydroxymethyl)propane-1,3-diol for a short amplicon PCR. Thus, by selecting suitable conditions of the lysis composition, in particular by selecting a suitable reactive compound and concentration of the reactive compound, the obtained nucleic acids can be adjusted to be suitable for certain nucleic acid analysis methods. details are described elsewhere herein.

According to one embodiment, the method further comprises analyzing the nucleic acids, wherein analyzing includes performing a next generation sequencing method. Next generation sequencing advantageously allows to determine the sequence of nucleic acids in a high throughput format. However, nucleic acids from fixed biological samples have often been associated with artifacts that are detrimental in next generation sequencing. The methods of the present invention advantageously provide high quality nucleic acids, which are particularly suitable for next generation sequencing as demonstrated by the examples. In embodiments, performing next generation sequencing according to the present disclosure may involve i. attaching a unique molecular identifier sequence to the nucleic acids, wherein each nucleic acid molecule comprises a different unique molecular identifier sequence; ii. amplifying the nucleic acids with the attached unique molecular identifier sequence; and iii. sequencing the nucleic acids. Such a method advantageously allows sequencing of the obtained nucleic acids having a low reads-per-unique molecular identifier sequence value, in particular less than 20, such as less than 15, less than 12, preferably 10 or less.

Further suitable nucleic acid analysis methods may be selected from or involve one or more of an amplification reaction, a polymerase chain reaction (PCR), isothermal amplification, reverse transcription polymerase chain reaction (RT-PCR), reverse transcription amplification, quantitative real time polymerase chain reaction (qPCR), DNA or RNA sequencing, reverse transcription, LAMP (loop mediated isothermal amplification), RPA (recombinase polymerase amplification), tHDA (helicase dependent amplification), NEAR (nicking enzyme amplification reaction) and other types of amplifications.

### The Use According to the Third Aspect

The third aspect of the invention pertains to the use of a proteolytic enzyme, preferably a protease, such as proteinase K, for performing a proteolytic digestion after lysing a fixed biological sample, said sample comprising crosslinks between nucleic acid molecules and protein molecules due to the fixation, wherein said prior lysis involves digestion with a proteolytic enzyme and heating the lysed sample to reverse crosslinks, preferably in a method according to the first or second aspect of the present invention. Details of the method according to the first and second aspect are also disclosed in claims 1 to 22 to which it is referred.

According to a preferred embodiment, the proteolytic enzyme is a protease, in particular a serine protease such as proteinase K. Suitable proteolytic enzymes and digestion conditions have been disclosed above in conjunction with the method of the first aspect, in particular in conjunction with step (a) and step (c) to which it is referred and the disclosure also applies here. Furthermore, suitable heating conditions and embodiments for reversing crosslinks have been described above in conjunction with the method according to the first aspect, in particular in conjunction with step (b). It is referred to the respective disclosure which also applies here.

### The Use According to the Fourth Aspect

The fourth aspect pertains to the use of a glycosylase, preferably a DNA glycosylase, more preferably a uracil DNA glycosylase, such as a uracil-N-glycosylase, for performing an enzymatic treatment, wherein the enzymatic treatment is completed in 30 min or less, 20 min or less, 15 min or less or 10 min or less. Preferably such use is within the method according to the first or second aspect of the present invention, more preferably between step (b) and step (c).

As is disclosed herein, such glycosylase treatment allows to remove artifacts caused by the fixation of the biological sample (such as caused by formalin crosslinking). Suitable glycosylases for performing such enzymatic treatment has been disclosed herein in conjunction with the method according to the first aspect, as well as suitable conditions for performing the enzymatic treatment using a glycosylase, such as suitable temperatures and incubation times, as well as suitable conditions within the reaction mixture. It is referred to the respective disclosure. Suitable conditions for performing such glycosylase treatment are also known in the art. Furthermore, the present disclosure discloses suitable lysis/digestion compositions that are compatible with such glycosylate treatment thereby allowing the in-process use of a glycosylase, such as uracil-N-glycosylase, to remove artefacts caused by the fixative induced crosslinking.

### The Method According to the Fifth Aspect

According to a fifth aspect of the invention, a method is provided for processing a biological sample, wherein the fixed biological sample comprises crosslinks between nucleic acid molecules and protein molecules due to the fixation, said method comprising
(a) lysing the fixed biological sample, wherein lysis involves digestion with a proteolytic enzyme, wherein lysing comprises preparing a lysis mixture, wherein the lysis mixture comprises (i) the fixed biological sample, and (ii) a lysis composition comprising the proteolytic enzyme and preferably a reactive compound, more preferably selected from Tris and spermidine;
(b) heating the lysed sample to reverse crosslinks;
(c) optionally, adding a proteolytic enzyme and performing a proteolytic digestion;
(d) purifying nucleic acids from the lysed sample; and
(e) analyzing the purified nucleic acids wherein analysis comprises amplifying nucleic acid molecules having a size of less than 500 nt, such as ≤300 nt, ≤200 nt, ≤150 nt or ≤100 nt.

Details with respect to steps (a) to (e) were already disclosed in conjunction with the methods according to the first and second aspect and it is referred to the above disclosure which also applied here. As disclosed above, optionally one or more additional treatment steps may be performed between steps (b) and (c) and this may be advantageous in order to prepare the nucleic acids for analysis step (e).

The purified nucleic acid may be DNA or RNA. RNA is preferably reverse-transcribed to cDNA prior to amplification in step (e). If the nucleic acid is a double-stranded molecule, such as a double stranded DNA molecule, what is preferred, the above indications with respect to the size (length) in “nt” refers to “bp”. Thus, if a double-stranded DNA molecule has a size of 150 nt, said double-stranded DNA molecule has a size of 150 bp.

In particular, nucleic acids having a small size can be purified and amplified by the method of the fifth aspect. The method according to the fifth aspect is in particular suitable for analyzing nucleic acid molecules having a short size in an amplification-based analysis method such as a PCR. As is demonstrated in the examples a short amplicon PCR of nucleic acids purified using the method according to the fifth aspect led to low Ct-values.

According to one embodiment, a reactive compound (e.g. a formalin scavenger, preferably selected from Tris and spermidine) is present in the lysis mixture and/or lysis composition used in step (a) in a concentration in the range of 3-100 mM, 5-50 mM or 10-25 mM. Such lower concentration of reactive compound (which may be a formalin scavenger such as Tris or spermidine) in the lysis composition/lysis mixture used in step (a) of e.g. 3-100 mM, preferably 5-50 mM, more preferably 10-20 mM, provides nucleic acid fragments of a size that is particularly suitable for performing a short amplicon amplification reaction. This was unexpected as the prior art considers a higher degree of fragmentation as disadvantageous for subsequent amplification reactions. However, the examples surprisingly show that the fragmentation of the purified DNA improves the performance of a downstream PCR when using a short amplicon PCR, by a very significant amount. Without wishing to be bound in theory, it is assumed that this effect is due to a greater accessibility of the nucleic acid (such as DNA) when it is fragmented strongly, potentially because these breaks occur at points where a crosslink was present in the fixed biological sample. As such crosslink points are inhibitory to the PCR, removing those points during the lysis procedure as applied in the method according to the fifth aspect improves the efficiency of the PCR reaction.

The lysis mixture in particular comprises a reactive compound as disclosed herein. Examples are disclosed in conjunction with the method according to the first aspect and it is referred to the respective disclosure which also applied here. In one embodiment, the reactive compound comprises a primary amine, and preferably is 2-amino-2-(hydroxymethyl)propane-1,3-diol or a derivative thereof. Other features of step (a) are disclosed above in conjunction with the method according to the first aspect and it is referred thereto.

The lysis composition used in step (a) may furthermore comprise
(i) a salt; and
(ii) a detergent.

Suitable salts and detergents for the lysis composition used in step (a) as well as suitable concentrations were already disclosed in conjunction with step (a) of the method according to the first aspect and it is referred to the respective disclosure.

The lysis composition and/or lysis mixture used in step (a) of the method according to the fifth aspect may have a pH in the range of 6.0 to 9.5, preferably 7.0 to 8.0.

The nucleic acids, which are advantageously released by performing method steps (a) and (b) and optionally, but preferably, step (c) are purified in step (d). Further details and embodiments of steps (b), (c) and (d) are disclosed herein, in particular in conjunction with the methods according to the first and second aspect, and it is referred to the respective disclosure which also applies here. Preferably, step (c) is performed in the method according to the fifth aspect. As disclosed in conjunction with the method according to the first aspect, one or more additional treatment steps may be performed between step (b) and step (c), such as a nuclease digestion and/or an enzymatic treatment step involving the use of a glycosylase, such as uracil-N-glycosylase. Details were described above in conjunction with the method according to the first aspect and it is referred to respective disclosure which also applies here.

### The Method According to the Sixth Aspect

According to a sixth aspect of the invention, a method is provided for processing a biological sample, wherein the fixed biological sample comprises crosslinks between nucleic acid molecules and protein molecules due to the fixation, said method comprising
(a) lysing the fixed biological sample, wherein lysis involves digestion with a proteolytic enzyme, wherein lysing comprises preparing a lysis mixture, wherein the lysis mixture comprises (i) the fixed biological sample, and (ii) a lysis composition comprising the proteolytic enzyme and preferably a reactive compound, more preferably selected from Tris and spermidine;
(b) heating the lysed sample to reverse crosslinks;
(c) optionally, adding a proteolytic enzyme and performing a proteolytic digestion;
(d) purifying nucleic acids from the lysed sample; and
(e) analyzing the purified nucleic acids wherein analysis comprises amplifying nucleic acid molecules having a size of at least 500 nt and/or a size of less than 500 nt.

Details with respect to steps (a) to (e) were already disclosed in conjunction with the methods according to the first and second aspect and it is referred to the above disclosure which also applied here. As disclosed above, optionally one or more additional treatment steps may be performed between steps (b) and (c) and this may be advantageous in order to prepare the nucleic acids for analysis step (e).

The purified nucleic acid may be DNA or RNA. RNA is preferably reverse-transcribed to cDNA prior to amplification in step (e). If the nucleic acid is a double-stranded molecule, such as a double stranded DNA molecule, what is preferred, the above indications with respect to the size (length) in “nt” refers to “bp”. Thus, if a double-stranded DNA molecule has a size of 500 nt, said double-stranded DNA molecule has a size of 500 bp.

The method according to the sixth aspect is in particular suitable for analyzing nucleic acids have a short size, such as less than 500 nt and/or a size of at least 500 nt in an amplification-based analysis method such as PCR, as is demonstrated by the Examples. In particular, nucleic acids having a small or large size can be obtained and amplified using the method of the sixth aspect. Without being bound to theory, it is believed that a concentration of reactive compound (e.g. Tris or spermidine) of at least 10 mM, in particular at least 20 mM, at least 40 mM, at least 60 mM or preferably at least 75 mM is highly effective in isolating nucleic acids having short sizes and large sizes and thus obtain more nucleic acids and/or nucleic acids of higher quality having a short size and large size.

In embodiments, the lysis mixture and/or lysis composition comprise 10-1000 mM reactive compound, in particular 20-500 mM, 50-300 mM, 75-250 mM or preferably 85-200 mM, such as about 100-150 mM. The lysis mixture in particular comprises a reactive compound as disclosed herein, e.g. Tris or spermidine. Suitable reactive compounds were disclosed in conjunction with the method according to the first aspect and it is referred to the respective disclosure which also applies here. The reactive compound may comprise a primary amine and preferably is 2-amino-2-(hydroxymethyl)propane-1,3-diol or a derivative thereof.

The lysis composition used in step (a) may furthermore comprise
(i) a salt; and
(ii) a detergent.

Suitable salts and detergents for the lysis composition used in step (a) as well as suitable concentrations were already disclosed in conjunction with step (a) of the method according to the first aspect and it is referred to the respective disclosure.

This may be further supported by providing a pH of 6.0 to 9.5, such as 7.0 to 9.0 or 8.0 to 9.0, in the lysis mixture and/or lysis composition.

The nucleic acids, which are advantageously released by performing method steps (a) and (b) and optionally, but preferably, step (c) are purified in step (d). Further details and embodiments of steps (b), (c) and (d) are disclosed herein, in particular in conjunction with the methods according to the first and second aspect, and it is referred to the respective disclosure which also applies here. Preferably, step (c) is performed in the method according to the sixth aspect. As disclosed in conjunction with the method according to the first aspect, one or more additional treatment steps may be performed between step (b) and step (c), such as a nuclease digestion and/or an enzymatic treatment step involving the use of a glycosylase, such as uracil-N-glycosylase. Details were described above in conjunction with the method according to the first aspect and it is referred to respective disclosure which also applies here.

In step (e), the nucleic acids are analyzed by a method comprising amplifying nucleic acids. Such amplification methods have been disclosed herein and it is referred thereto. In particular, nucleic acids may be amplified using a polymerase enzyme. According to one embodiment, the nucleic acids are amplified using a PCR, in particular a PCR wherein short nucleic acid molecules are amplified (also referred to as short amplicon PCR) and/or a PCR wherein large nucleic acid amplified are amplified (also referred to as large amplicon PCR). Advantageously, short and large nucleic acid molecules can be amplified using this method. By adapting the concentration of the reactive compound (e.g. selected from Tris and spermidine) in the lysis composition/lysis mixture, one can control the fragment size. According to one embodiment, step (e) analysis comprises amplifying nucleic acid molecules having a size of at least 500 nt, such as at least 550 nt, at least 600 nt, at least 650 nt or at least 700 nt.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

As used in the subject specification and claims, the singular forms “a”, “an” and “the” include plural aspects unless the context clearly dictates otherwise. The terms “include,” “have,” “comprise” and their variants are used synonymously and are to be construed as non-limiting. Further components and steps may be present. Throughout the specification, where compositions are described as comprising components or materials, it is additionally contemplated that the compositions can in embodiments also consist essentially of, or consist of, any combination of the recited components or materials, unless described otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

### EXAMPLES

The following examples are for illustrative purpose only and are not to be construed as limiting this invention in any manner. They demonstrate that the DNA extraction from fixed samples can be advantageously improved by performing an additional proteinase digestion step after a first proteinase digestion step and a crosslink removal step. This second proteinase digestion step improves the extraction process and provides pure DNA of high quality. The DNA can be purified with high yield, good fragment size and better suitability for downstream PCR amplification (such short and large amplicon PCR). The purified DNA is also highly advantageous for Next Generation Sequencing (NGS) applications, as a particular low read-per-UMI (=Unique Molecular Identifier, also referred to as Unique Molecular Index) value can be obtained.

Moreover, the examples show that the size of the DNA fragments can be modified by adaption of the lysis composition. In addition, the processing time required for a uracil-N-glycosylase (UNG) treatment step, if performed, can be significantly reduced compared to prior art methods.

Throughout the examples, DNA is extracted from FFPE tissue samples. After deparaffinizing the FFPE tissue sample, the sample is lysed. The sample material in the lysis composition (corresponding to sample+lysis solution) is subjected to a proteinase K digestion step, followed by a crosslink removal step applying heating. Afterwards, the lysed sample can be subjected to an enzymatic treatment such as an RNase and/or UNG treatment. Then, a (second) proteinase K digestion step is performed, which removes protein and peptides still crosslinked to the DNA. Afterwards, the DNA is purified from the digested sample, e.g. by binding the DNA to a solid phase, followed by wash cycles and elution of the bound DNA. The purified DNA is then ready for further use and analysis.

### 1. Example 1: Improvements by Performing an Additional Proteinase Digestion Step

### 1.1. DNA Yield, Degree of Fragmentation and Impact on Downstream PCR Performance are Improved

(a) Materials and Methods

FFPE Tissue Samples

Various human FFPE tissue samples were used in the present example, including prostate, lung, kidney, spleen and breast cancer. 10 μm sections were cut from FFPE blocks using a Leica rotary microtome and two or three 10 μm sections were applied per preparation.

Extraction Protocol

DNA was extracted from FFPE tissue according to the following protocol:
1. FFPE tissue sample preparation
   400 μL Deparaffinization Solution (DPS) was added to the FFPE tissue samples and vortexed.
   Samples were incubated for 3 min at 56° C.
2. Lysis of FFPE tissue sample
   Lysis is assisted by using a lysis solution, such as a lysis buffer. Preferably, the lysis solution comprises a detergent, salt and a buffer and such lysis buffer was used in this example. As detergent, an anionic detergent such as SDS may be 0 used. The salt may be a non-buffering salt such as an alkali metal salt. A chloride salt such as NaCl or KCl may be used. The pH of the lysis buffer may be in the range of 6.0-9.5, such as 7.0 to 9.0. Tris may be used as buffer. The lysis solution may optionally comprise a chelating agent such as EDTA to inhibit nucleases such as DNases. In embodiments, the lysis solution is a lysis buffer comprising at least 0.1% (w/v) detergent, at least 300 mM or at least 500 mM salt and a buffering agent. The lysis buffer used may comprise 0.1-0.5% (w/v) detergent (e.g. anionic detergent such as SDS), 400-800 mM salt (such as an alkali metal halide, e.g. KCl or NaCl) and a buffering agent (e.g. Tris). Such lysis buffers are known in the art. In this example a commercially available lysis buffer was used (FTB, QIAGEN). In 0 this example, the lysis composition was prepared by mixing the lysis buffer, proteinase K (in a solution comprising 20 mg/ml proteinase K) and water. In one embodiment, Tris was added to the lysis composition (referred to as “high Tris”; the lysis compositions without added Tris are referred to as “low Tris”). As described herein, Tris can act as a formaldehyde scavenger and may support the resolution of the cross links.
   A lysis composition as defined below was prepared and added to the FFPE samples treated according to step 1:

| | Lysis | 1M Tris | | |
|---|---|---|---|---|
| Lysis type | Buffer | pH 9 | Prot.-K | Water |
| “High Tris” | 25 μL | 10 μL | 20 μL | 45 μL |
| “Low Tris” | 25 μL | — | 20 μL | 55 μL |

Samples were incubated for 1 h at 56° C. and shaken in a thermoshaker for the proteinase digestion (using proteinase K).
Samples were then incubated for 1 h at 90° C. without shaking to reverse cross-links. Such heating step also inactivates the proteinase.
3. Uracil-N-Glycosylase (UNG) digestion
   The blue DPS phase was removed from on top of the aqueous phase which is the lysate, or the lower clear phase was transferred to a new tube.
   Samples were further diluted for digestion with Uracil-N-Glycosylase (UNG), using 115 μL water and 35 μL UNG (1 U/μL).
   The samples were then incubated for 5 min at 50° C. without shaking.
4. RNase A digestion
   4 μL RNase A were added per sample.
   The samples are mixed and incubated for 2 min at room temperature.
5. Additional (second) proteinase digestion step
   5-20 μL proteinase K were added.
   The samples are mixed and incubated for 15 min at 65° C. at 450 rpm in a thermoshaker.
   Nucleic acids such as DNA are then purified from the digested sample. Here, any suitable purification method can be used, as the nucleic acids are rendered well-accessible by the above described digestion/pretreatment protocol.
6. Purification of DNA
   250 μL buffer AL (QIAGEN) and 96-100% ethanol were added respectively and the samples were mixed.
   The lysate was transferred to a QIAamp® MinElute spin column, followed by centrifugation and discard of the flow-through.
   500 μL buffer AW1 (QIAGEN) was added, followed by centrifugation and discarding of the flow-through.
   500 μL buffer AW2 (QIAGEN) was added, followed by centrifugation and discarding of the flow-through.
   500 μL 96-100% ethanol was added, followed by centrifugation and discarding of the flow-through and a dry spin at full speed.
   30 μL or 50 μL elution buffer ATE (QIAGEN) was applied onto the membrane, followed by centrifugation. The extracted and purified DNA was in the obtained flow-through.

Control

For comparison, the protocol above was performed except for the additional proteinase K digestion step (see above step 5.), which was omitted.

b) Analysis of the DNA Yield

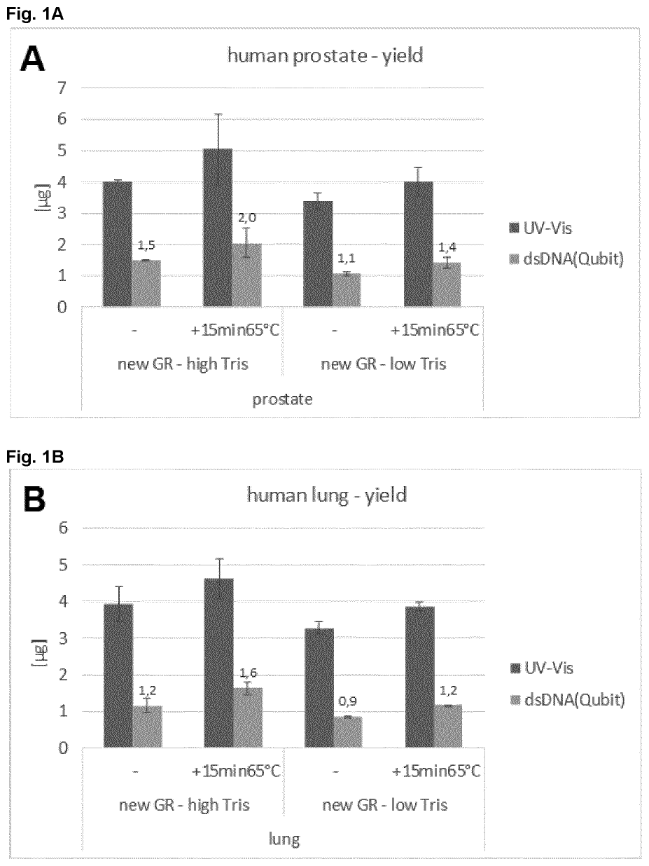

In this set of experiments, the DNA yield of the extract using the above disclosed protocol was determined using the QIAxpert and Qubit instruments. The obtained DNA yields using either instrument are shown in FIGS. 1A-1E (UV-Vis=QIAxpert; dsDNA(Qubit)=Qubit).

Example 1.1 demonstrates that performing an additional proteinase digestion step after the crosslink removal step at high temperatures improves the DNA yield. Moreover, DNA fragments having a larger average size are obtained, which is particularly advantageous for obtaining DNA suitable for downstream PCR within a large amplicon PCR.

The second proteinase digestion step resulted in higher yields as measured with UV-Vis and by fluorometric determination (Qubit) compared to the control protocol without the additional proteinase K digestion step (see FIGS. 1A-1E). The higher DNA yield was obtained for all tested FFPE tissue samples and lysis solutions. Generally, it is considered in the art that the protein digestion in the sample is essentially complete after the initial proteinase digestion step, as carried out by the conventional FFPE extraction methods (e.g. 1 h proteinase K, 56° C.). Thus, it was highly surprising that performing a second proteinase digestion step after the heat assisted cross-link removal step significantly improves the results. Without wishing to be bound in theory it is assumed that the cross-links present in the fixed sample can cause particularly persistent protein associations with nucleic acids (such as DNA), which can potentially protect the protein during the first proteinase digestion step. Furthermore, steric effects are possible that render protein inaccessible to the proteinase during the initial proteinase digestion step. After the cross-linking reversal step at high temperature (e.g. at least 85° C. or at least 90° C.), these associations are weakened and/or the sample is denatured sufficiently that remaining protein becomes accessible and can be efficiently removed during the second proteinase digestion step.

FIGS. 1A-1E further show that for some FFPE tissue types the high Tris lysis composition tested led to higher DNA yields than the low Tris lysis composition (see prostate, lung; FIGS. 1A and 1B). On the other hand, for kidney tissue the low Tris lysis composition resulted in higher DNA yields than the high Tris lysis composition (see FIG. 1C), while for spleen and breast cancer tissue no difference was observed (see FIGS. 1D and 1E). Therefore, modifying the Tris concentration in the lysis composition can be advantageously used to further improve the DNA yield depending on the used tissue type.

The additional proteinase K digestion step advantageously increases the DNA yield and thus improves the extraction of DNA from various FFPE tissue sample types.

(c) Analysis of the Degree of Fragmentation of the Extracted DNA

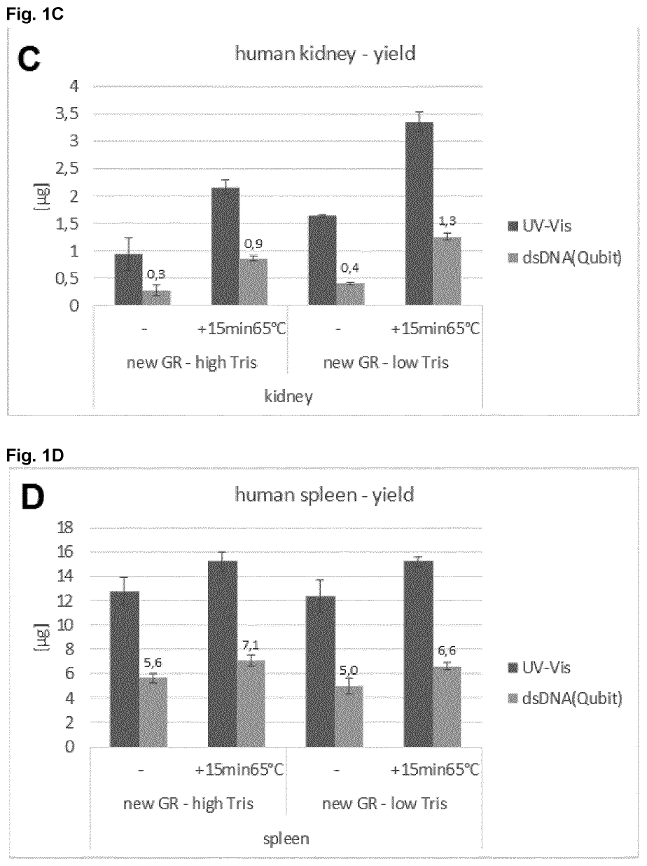

In this experiment, the degree of fragmentation was analyzed using gel electrophoresis. As sample material, the above extracted DNA was used from human kidney and breast cancer FFPE tissue. The obtained results are shown in FIG. 2.

The gel electrophoresis in FIG. 2 shows that the additional proteinase digestion step leads to an increase in the average size of the DNA fragments (bands are shifted; see samples with “+2.PK lysis 15′65° C.”). This increase was observed for both FFPE tissue types and in both lysis solution, high Tris and low Tris. This result was surprising and not expected. As an increase in fragment size of the extracted DNA can be advantageous for downstream PCR applications, using the additional proteinase digestion after the heat-assisted cross-link removal step improves the DNA extraction.

(d) Impact on Downstream PCR Performance

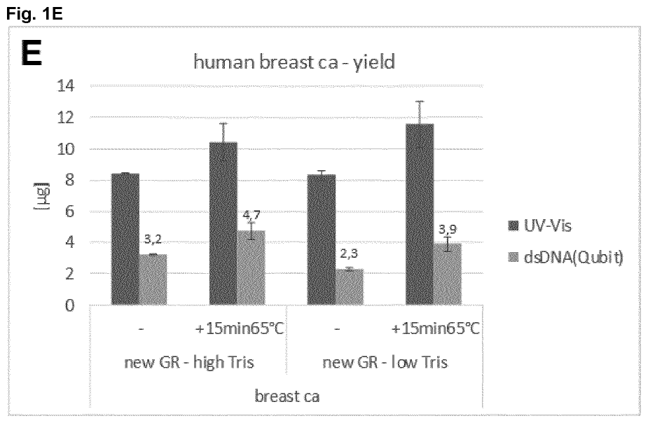

In this example, the extracted DNA was analyzed by PCR to determine, whether the PCR performance is affected by the increase in size of DNA by the additional proteinase K step. A quantitative Real Time PCR was performed and the Cq values were determined. “Cq” values can be interchangeably used with “Ct” values. Cq values are inversely proportional to the original relative amount of the extracted DNA. Both short amplicon PCR with 66 bp fragments and large amplicon PCR with 500 bp fragments were performed. The obtained results are shown in FIG. 3 (the dark shaded columns correspond to same amount of DNA per reaction mixture and the light shaded columns correspond to same volume of diluted eluate per reaction mixture).

As shown in FIG. 3, DNA extracted using the additional proteinase K digestion step overall reduces the Cq value and thus improves the PCR performance. This improvement was observed for both tested FFPE tissue types (human breast cancer and kidney tissue) and both lysis solution (high and low Tris). Hence, the improvement in quality of the extracted DNA also leads to better PCR results, particularly with large amplicons (500 bp). The results indicate that a larger amount of previously inaccessible long DNA strands has been made accessible to the PCR reaction by performing a second proteinase digestion after the de-crosslinking step.

(e) Further Conclusions

An additional proteinase digestion step after the crosslink removal step enhances the DNA extraction from various FFPE tissue samples. In particular, DNA is extracted with a higher yield and with a larger fragment size, as well as a higher quality in respect to PCR amplification for short and large amplicons. Therefore, the additional proteinase digestion step is highly advantageous for DNA extraction from fixed biological samples, such as FFPE samples but also fixed liquid samples. As shown, one or more further enzymatic digestion steps may be performed in-between the de-crosslinking step and the second proteinase step.

1.2. Next Generation Sequencing (NGS) Performance is Enhanced

Example 1.2 demonstrates the improvements of DNA extraction from FFPE tissue samples by performing an additional proteinase digestion step according to the present disclosure. As noted, Proteinase K may be used as proteinase. By using the extracted DNA of Example 1.1 for NGS, very high NGS performance was measured. In particular, a low reads-per-UMI value of below 10 was measured for all tested tissue samples.

(a) Materials and Methods

DNA extracted from human FFPE tissue samples, including prostate, lung, kidney, and breast cancer as described in Example 1.1 were used for NGS performance analysis.

Sample Preparation and Sequencing Workflow

The extracted DNA was used as a template for a sequencing library. The QIAseg™ Targeted DNA Panel Handbook (QIAGEN, 05/2017) was followed using the QIAseg™ Targeted Panel for Illumina Instruments protocol. As QIAGEN targeted DNA Panel the Human Lung Cancer Panel with UMI technology was used. The UMI technology is based on integration of unique molecular identifier (UMI) sequences (also referred to as Unique Molecular Index) into a single gene-specific, primer-based targeted enrichment process, which overcomes biases/artifacts of the DNA polymerase and amplification processes:
Sequence reads having different UMIs represent different original molecules.
Sequence reads having the same UMIs are the result of PCR duplication from one original molecule.

Errors from PCR amplification and from the sequencing process may also be present in final reads that lead to false positive variants in sequencing results. These artifact variants can be greatly reduced by calling variants across all reads within a unique UMI instead of picking up variants at the original read level.

(b) Performance in NGS

As discussed above, each molecule of double stranded DNA is tagged with an UMI barcode prior to amplification. This allows to distinguish truly unique molecules that were detected in NGS from PCR amplicons.

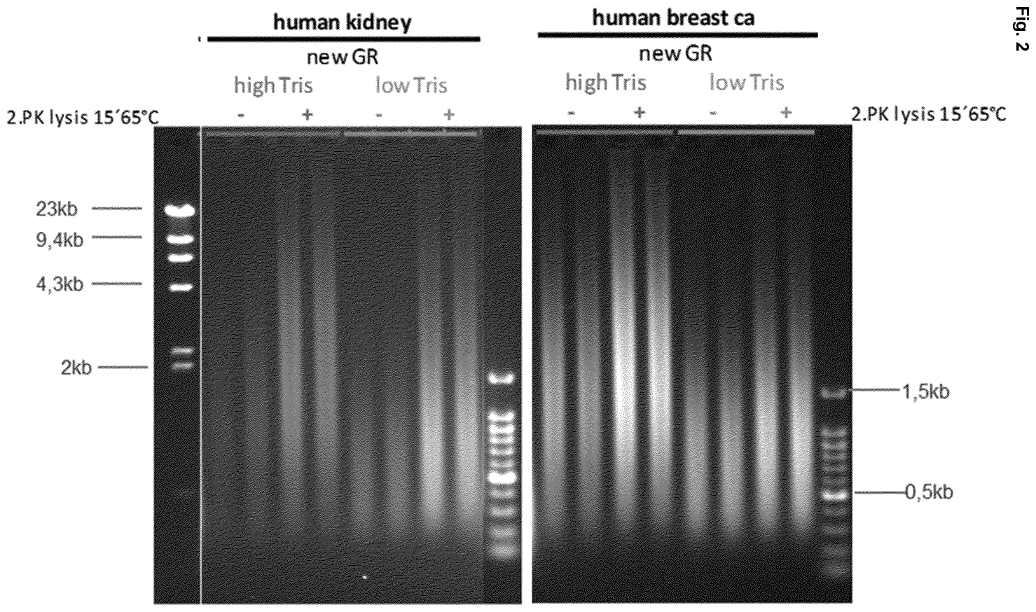

In FIG. 4 the detected reads per UMI are plotted for the extracted DNA. A reads-per-UMI value over 10 indicates that the same molecule was read more than 10 times, and is an indication of too much overamplification/not enough complexity in the starting material.

As shown in FIG. 4, the extraction method according to the present disclosure shows a very high NGS performance for all samples, as a low reads-per-UMI of below 10 was measured. This demonstrates that the method of the present disclosure is advantageous for extraction of DNA from various FFPE sample types. Moreover, the additional/second Proteinase K step improves the NGS performance, in particular for prostate, kidney and in some cases also for lung and breast cancer tissue (for high Tris lysis composition, see FIG. 4 “GR-high Tris”).

Overall, the NGS performance is very high when using the purification method according to the present disclosure.

### 2. Example 2: Comparison of DNA Extraction Using the Method of the Present Disclosure with Prior Art Methods

Example 2 further demonstrates the improvements of DNA extraction from FFPE tissue samples by using an additional proteinase K digestion step according to the present disclosure. In line with Example 1.1, a high DNA yield was obtained. Moreover, larger fragment size and better PCR performance was measured. Importantly, also the NGS performance was significantly enhanced compared to prior art methods.

(a) Materials and Methods

FFPE Tissue Samples

Human atrium FFPE tissue was used in the present example. 10 μm sections were cut from FFPE blocks using a Leica rotary microtome and two 10 μm sections were applied per preparation.

Extraction Protocol

The extraction protocol of Example 1.1 was followed in the present Example.

Control

In line with Example 1.1, the additional proteinase K digestion step (see above step 5.) was omitted in the control for comparison. Moreover, an additional control was performed for comparison by performing the first proteinase K step over night at 56° C. and omitting the additional proteinase K digestion step (“o/n 56° C.”).

Reference Protocols

As reference protocols (i.e. prior art methods), the Maxwell® RSC DNA FFPE Kit Technical Manual (Promega, Revised 11/17) was followed, as well as the Maxwell® RSC FFPE Plus DNA Kit Technical Manual (Promega, Revised 12/19). Moreover, as reference protocol, the QIAamp® DNA FFPE Tissue Handbook (QIAGEN, June 2012) was followed using the QIAGEN supplementary protocol on “Purification of genomic DNA from FFPE tissue using the QIAamp® DNA FFPE Tissue Kit and Deparaffinization Solution”.

b) Analysis of the DNA Yield

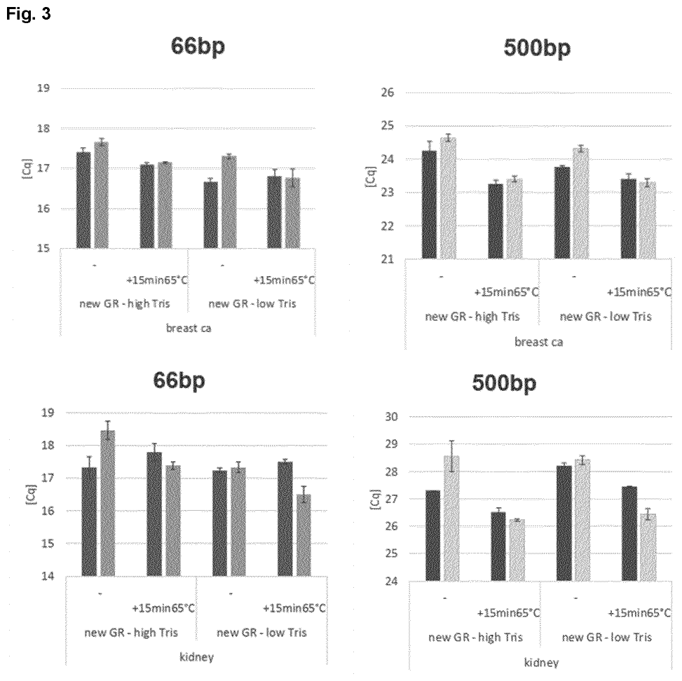

In this set of experiments, the DNA yield of the extract was determined using the QIAxpert and Qubit instruments. The obtained DNA yields using either instrument are shown in FIGS. 5A and 5B for human lung cancer tissue and human atrium, respectively (UV-Vis=QIAxpert; dsDNA(Qubit)=Qubit).

The additional proteinase K digestion step at 65° C. after the cross-linking step resulted in higher yields as measured with UV-Vis and by fluorometric determination (Qubit) compared to the control protocol without the additional proteinase K digestion step (see FIGS. 5A and 5B). The higher yield was obtained for both tested FFPE tissue types and both lysis compositions (high and low Tris). Importantly, performing the first proteinase K step longer as in the over night 56° C. control sample (see “o/n 56° C.”), did non result in a higher yield. Indeed, performing the second proteinase K step after the de-crosslinking step led to higher yields compared to the o/n 56° C. control, demonstrating that it is not the protein digestion time as such that is important but the particular sequence of steps as performed in the method according to the present disclosure, wherein the second proteinase digestion step is performed after the de-crosslinking step.

FIGS. 5A and 5B further show that the low Tris lysis composition resulted in even higher DNA yields for both tested tissue types. Therefore, for these tissue types a low Tris lysis composition (e.g. wherein the Tris concentration in the lysis composition is less than 50 mM, less than 30 mM, less than 25 mM, optionally in a range of 1 mM to 20 mM) may be used to further increase the yield. Significantly higher yields than the reference protocols were obtained.

(c) Analysis of the Degree of Fragmentation of the Extracted DNA

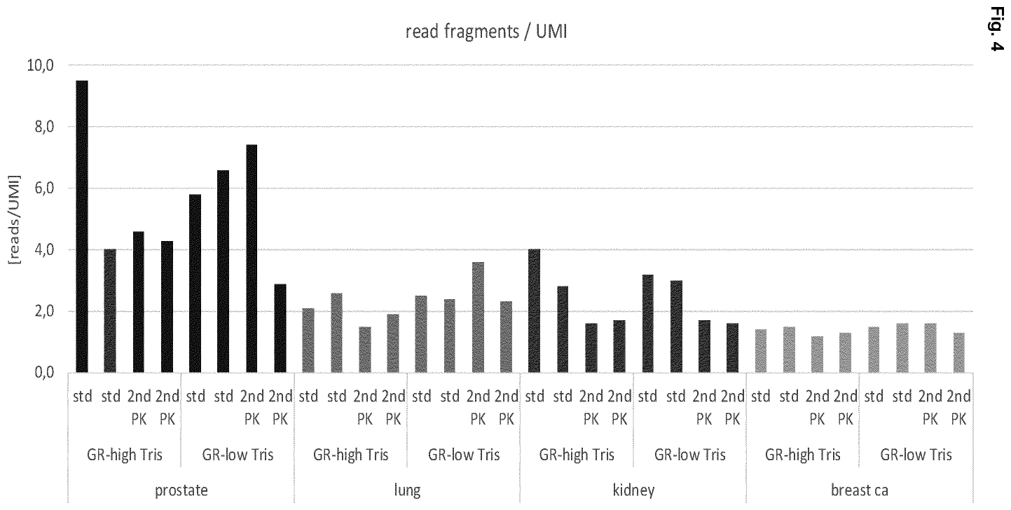

The degree of fragmentation was analyzed using gel electrophoresis. As sample material, the above extracted DNA was used from human lung cancer and atrium FFPE tissue. The obtained results are shown in FIGS. 6A and 6B, respectively.

The gel electrophoresis in FIGS. 6A and 6B show that the additional proteinase K digestion step leads to an increase in the average size of the DNA fragments (bands are shifted; see samples with “+2.PK lysis 15′65° C.”). This increase was observed for both FFPE tissue types and in both lysis compositions used. In line with the discussion on higher yield, this result was surprising and not expected. In comparison to the reference protocols, overall larger fragment sizes were obtained by performing the second proteinase K step according to the present disclosure. A larger fragment size can be advantageous for downstream PCR performance.

(d) Impact on Downstream PCR Performance

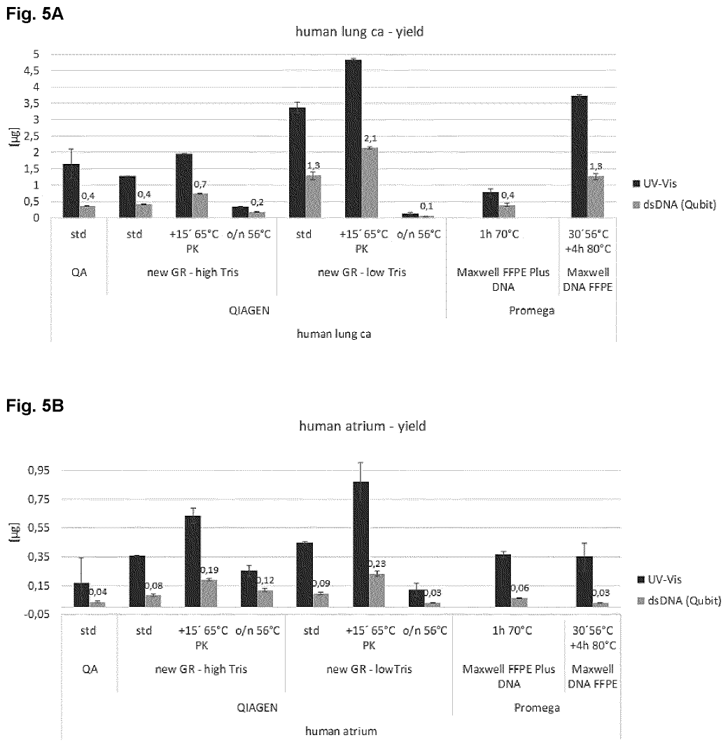

In this example, the extracted DNA was analyzed by PCR to determine, whether the PCR performance is affected by the increase in size of DNA by the additional proteinase K step. As before, a quantitative Real Time PCR was performed and the Cq values were determined. Both short amplicon PCR with 66 bp fragments and large amplicon PCR with 500 bp fragments were performed. The obtained results are shown in FIGS. 7A and 7B for short and large amplicons, respectively (the dark shaded columns correspond to same amount of DNA per reaction mixture and the light shaded columns correspond to same volume of diluted eluate per reaction mixture).

As shown in FIGS. 7A and 7B, DNA extracted using the additional proteinase digestion step overall reduces the Cq value and thus improves the PCR performance, in particular for large amplicon PCR with 500 bp fragments. The method according to the present disclosure using the additional/second proteinase digestion step leads to overall low Cq values, in particular the short amplicons compared to the reference protocols. Also the control sample, wherein a 56° C. over night proteinase K digestion step was overall outperformed by the method according to the present disclosure using the additional/second proteinase digestion step. The results indicate that a larger amount of previously inaccessible long DNA strands has been made accessible to the PCR reaction by performing the additional proteinase digestion after de-crosslinking. Moreover, the results indicate that not the total amount of digestion time is important to achieve a better PCR performance but the sequence of steps as performed in the method according to the present disclosure.

(e) Performance in NGS

In this example, the performance of the extracted DNA for NGS analysis was investigated. DNA was extracted from human atrium FFPE tissue samples using the above described extraction protocol (see Example 1.1. for used lysis compositions), with/without the additional proteinase digestion step (using proteinase K as proteinase).

The extracted DNA was used as a template for a sequencing library. The sample preparation and sequencing workflow described in Example 1.2 was followed and the reads-per-UMI values were determined as above.

Results

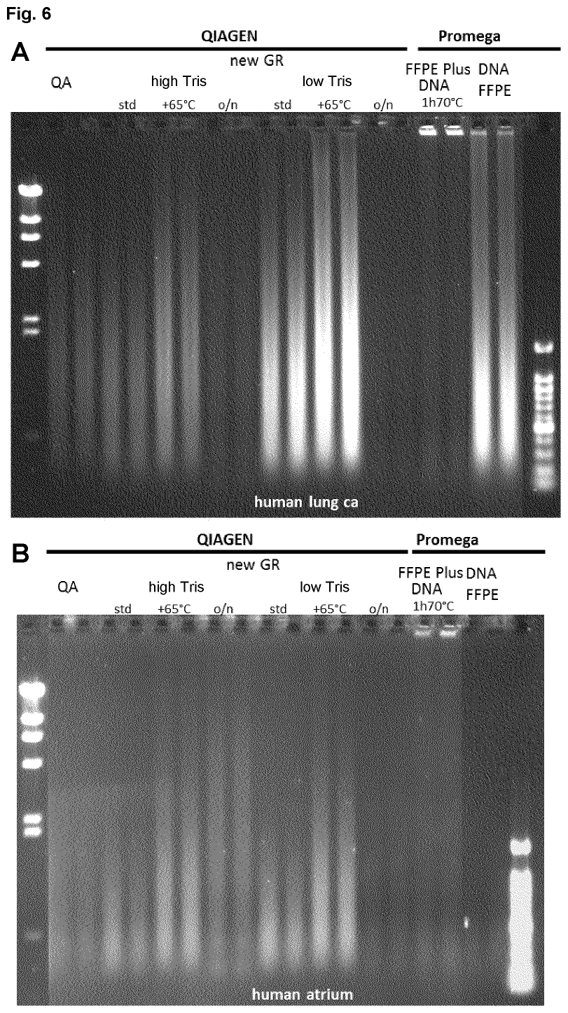

Each molecule of double stranded DNA is tagged with a unique molecular identifier (UMI) barcode prior to amplification. This allows to distinguish truly unique molecules that were detected in NGS from PCR amplicons. In FIG. 8 the detected reads per UMI are plotted for the extracted DNA. A reads-per-UMI value over 10 indicates that the same molecule was read more than 10 times, and is an indication of too much overamplification/not enough complexity in the starting material.

As shown in FIG. 8, the extraction method according to the present disclosure shows better performance than the QIAamp® FFPE DNA kit (“QA FFPE”) as well as the Maxwell RSC DNA FFPE or Maxwell RSC FFPE Plus DNA kits from Promega. All of those kits have UMI values far in excess of 10. The values of the DNA extracted by the method according to the present disclosure are all below a value of 10. The additional proteinase K incubation step led to even lower reads-per-UMI values.

Overall, the NGS performance is significantly improved by the extraction method according to the present disclosure, which can be performed fast and provides excellent result.

(e) Conclusions

This example demonstrates that performing an additional proteinase digestion step after a first proteinase digestion step and de-crosslinking step improves the quality of the purified nucleic acid, as shown based on DNA. The additional proteinase digestion step in particular increases the yield and fragment size, improves PCR performance and importantly, significantly enhances the NGS performance compared to the prior art methods. As discussed above, these results are highly surprising and unexpected. Moreover, by comparing the method according to the present disclosure comprising the additional/second proteinase digestion step with a first overnight 56° C. proteinase K digestion step, it was shown that not the overall digestion time is the cause of the better performance but the particular sequence of steps as performed in the method according to the present disclosure.

### 3. Example 3: Influence of DNA Extraction Using a Diluted Lysis Composition

Example 3 demonstrates that the lysis composition affects the DNA extraction from FFPE tissue samples. Dilution of the lysis composition allows to control the fragmentation of DNA in the crosslink removal step. Moreover, the example shows that by controlling the fragmentation shorter DNA fragments can be obtained that improve the performance of downstream PCR when using short amplicon PCR.

(a) Materials and Methods

FFPE Tissue Samples

To analyse the impact of the lysis composition in frame of a DNA extraction protocol, two different FFPE tissue samples were used: heart tissue and lung tissue, both from rat. 10 μm sections were cut from FFPE blocks using a Leica rotary microtome and one 10 μm section was applied per preparation.

Extraction Protocol

An extraction protocol as described in Example 1.1 was used, with the following modifications:
The lysis composition in the 2. step was prepared by combining 25 μL lysis buffer (FTB, QIAGEN), 20 μL proteinase K and 55 μL water (corresponding to the “low Tris” buffer used above for Example 1.1).
In step 3 the UNG was substituted with water and incubation was performed for 60 min at 50° C.
Step 5 was omitted (i.e. no second proteinase K digestion step).

Reference Protocol

As reference protocol, the QIAamp® DNA FFPE Tissue Handbook (QIAGEN, June 2012) was followed using the QIAGEN supplementary protocol on “Purification of genomic DNA from FFPE tissue using the QIAamp® DNA FFPE Tissue Kit and Deparaffinization Solution”. For lysis, 180 μL buffer ATL (QIAGEN) was mixed with 20 μL proteinase K.

(b) The Degree of Fragmentation May be Controlled by the Choice of the Lysis Solution in the Crosslink Removal Step

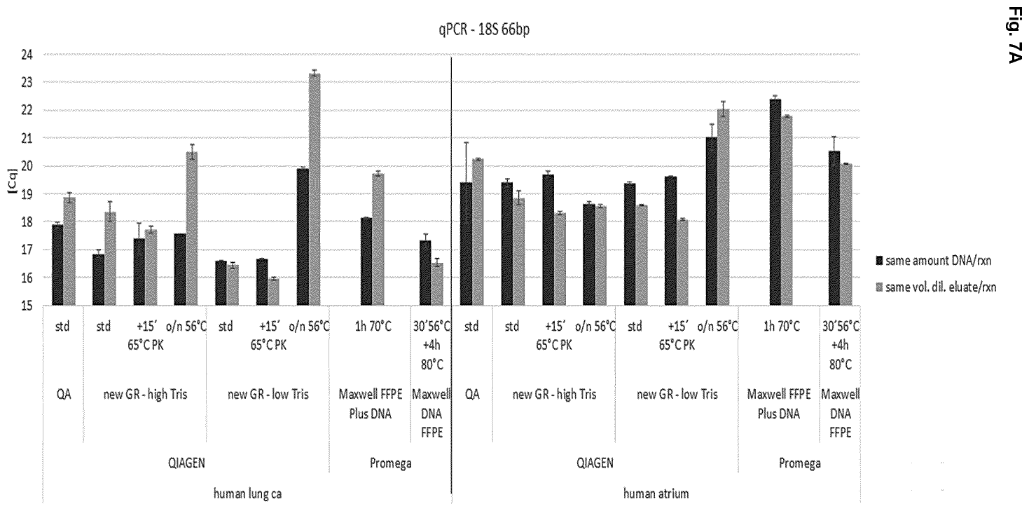

In the first set of experiments, the DNA extracted from the FFPE tissue samples was analyzed by gel electrophoresis (see FIG. 9). Thereby, the size distribution of the extracted DNA is visualized and conclusions can be drawn on the degree of fragmentation.

As shown in FIG. 9, a greater fragmentation of DNA was observed using the extraction method with the diluted lysis composition (see “GR std”=diluted lysis composition; “QA std”=reference lysis composition; L1-L3=DNA ladders as shown in FIG. 9). The diluted lysis composition for lysis and cross-link removal (see “GR std”) was used to allow the activity of Uracil-N-Glycosylase within the lysate, after a further 2.5 fold dilution. It was found that this led to smaller average sizes of extracted DNA compared to the reference protocol (see “QA std”). The smaller average size of extracted DNA was obtained for both FFPE tissue samples, the rat heart and rat lung.

Overall, a greater fragmentation of DNA was observed resulting from the extraction method with the diluted lysis composition. Therefore, a dilution of compounds present in the lysis solution allows to control the degree of fragmentation. These compounds in particular include Tris.

(c) The Performance of Downstream PCR is Affected by Fragmentation of the Extracted DNA

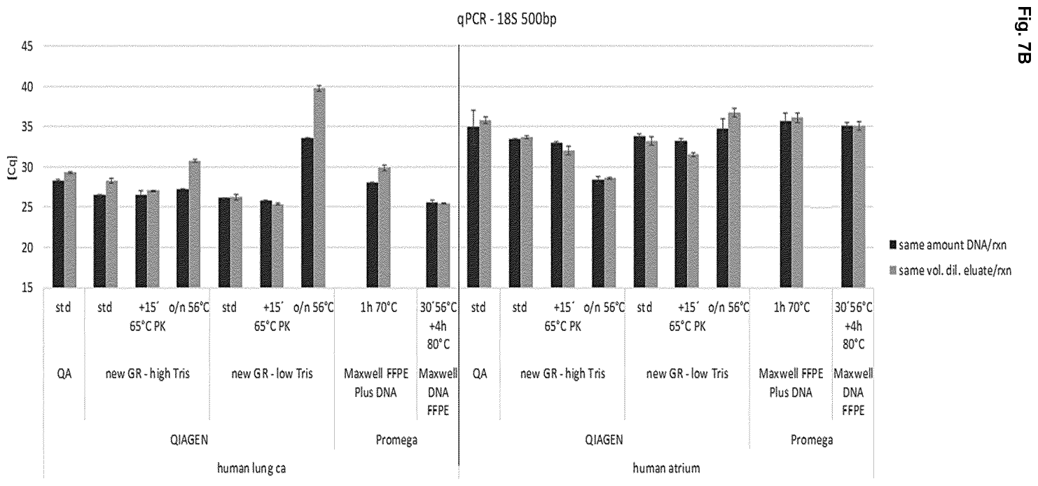

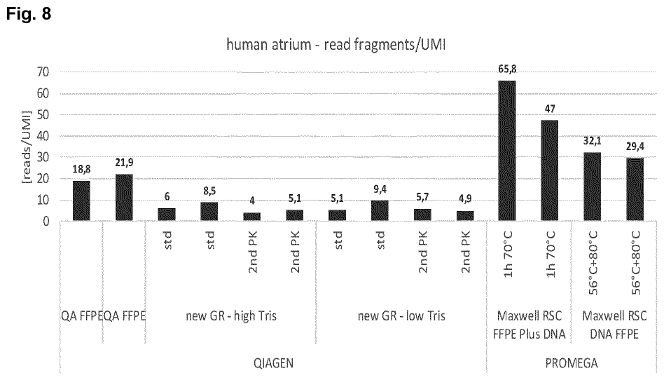

In this set of experiments, the influence of the DNA fragmentation on PCR amplification was analyzed. It was a particular object to investigate the quality of the extracted DNA in respect to the suitability to be amplified by PCR. As sample material, the DNA extracted above from the FFPE tissue samples was used. As before, a quantitative Real Time PCR was performed and Cq values were determined. The obtained Cq-values are plotted in FIGS. 10 and 11.

It is generally believed that a lower molecular weight and thus average length of DNA (a more fragmented DNA) is less reliable for downstream analyses. As shown in FIG. 10, the performance of PCR using long amplicons (727 bp) was slightly worse when using the more fragmented DNA obtained by the extraction method using the diluted lysis composition. This is reflected by the higher Cq value (“Fragmented”; about 23.6) in comparison to the reference protocol (“Standard”; Cq value of about 23.2). This is due to the fact that there are less fragments available having the minimum length required to amplify the large PCR product when the DNA is more fragmented.

However, surprisingly the more fragmented DNA obtained by the diluted lysis composition improved the performance of downstream PCRs when using short amplicon PCR (78 bp), by a very significant amount (see FIG. 11). While the reference protocol (“Standard”) has a Cq value of about 23, the obtained DNA sample from the diluted lysis composition (“Fragmented) has reduced Cq value of about 21.5. This was unexpected. Without wishing to be bound in theory it is assumed that this effect is due to a greater accessibility of the DNA when it is more fragmented, potentially because these breaks occur at points where a crosslink was present. As such crosslink points are inhibitory to the PCR, removing those points appear to improve the efficiency of the PCR reaction.

(d) Further Conclusions

Using an extraction method with the diluted lysis composition led to higher DNA fragmentation and thus smaller average DNA size. This could be due to the lower concentrations of the compounds used to assist the lysis, such as in particular the detergent, salt and optionally the chelating agent. Despite the smaller fragment size, the extracted DNA demonstrated an improved performance in downstream PCR when using short amplicon PCR. In this respect, a higher quality DNA sample was obtained.

### 4. Example 4: Impact of Additives in the Lysis Solution on Extracted DNA and UNG Incubation

Example 4 demonstrates that the addition of certain compounds to the lysis composition can advantageously be used to control the degree of fragmentation of DNA extracted from FFPE tissue samples. Therefore, the extraction protocol can be tuned to optimize it for either short or long fragments used in the downstream PCR. Moreover, Example 4 demonstrates that the method of the present disclosure advantageously allows to reduce the processing time. In particular, the uracil-N-glycosylase (UNG) treatment step can be reduced in incubation from 60 min to only 5 min.

FFPE Tissue Samples

In the present example, FFPE tissue samples from rat kidney and rat lung were used. 10 μm sections were cut from FFPE blocks using a Leica rotary microtome and two 10 μm section were applied per preparation.

Extraction Protocol

The extraction protocol of Example 1.1 above was followed without step 5 (i.e. no second proteinase K digestion step). For lysis of the FFPE tissue sample, following lysis compositions were prepared by mixing with the lysis buffer (FTB, QIAGEN):

| Additive | | | | | | |
|---|---|---|---|---|---|---|
| Type | Concentration | Volume | FTB Buffer | 1M Tris pH 9 | Prot.-K | Water |
| spermidine | 200 mM | 1.25 μL | 25 μL | 10 μL | 20 μL | 43.75 μL |
| spermin | 200 mM | 1.25 μL | 25 μL | 10 μL | 20 μL | 43.75 μL |
| DTT | 250 mM | 2 μL | 25 μL | 10 μL | 20 μL | 43 μL |
| glycin | 1 M | 10 μL | 25 μL | 10 μL | 20 μL | 35 μL |
| — | — | — | 25 μL | 10 μL | 20 μL | 45 μL |
| spermidine | 200 mM | 1.25 μL | 25 μL | — | 20 μL | 53.75 μL |
| spermin | 200 mM | 1.25 μL | 25 μL | — | 20 μL | 53.75 μL |
| DTT | 250 mM | 2 μL | 25 μL | — | 20 μL | 53 μL |
| glycin | 1 M | 10 μL | 25 μL | — | 20 μL | 45 μL |
| — | — | — | 25 μL | — | 20 μL | 55 μL |

Reference Protocols

As reference protocol, the GeneRead™ DNA FFPE Handbook (QIAGEN, March 2014) was followed, comprising a 60 min UNG treatment step.

(a) The Degree of Fragmentation can be Controlled by Additives in the Lysis Composition

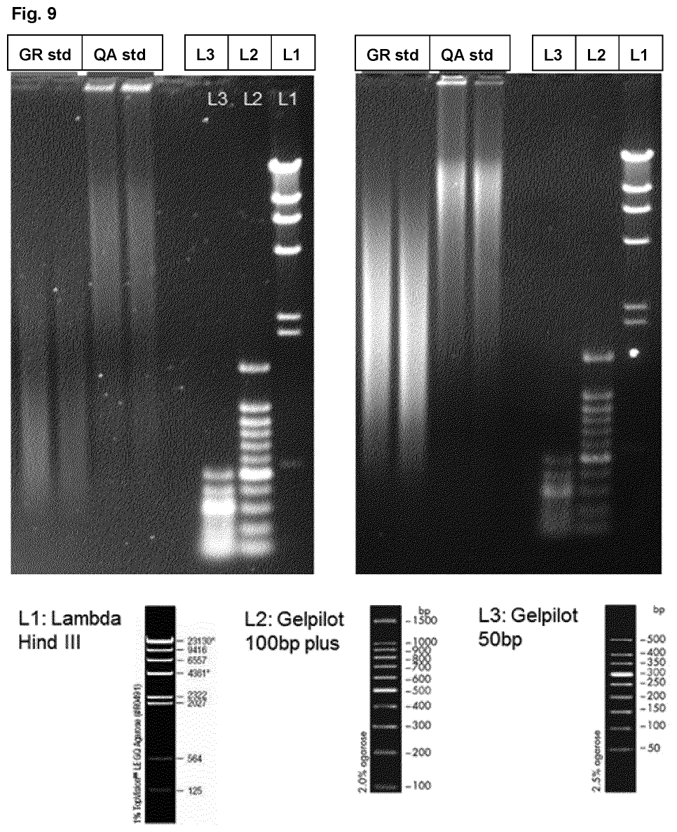

The fragmentation was analysed by gel electrophoresis of the extracted DNA samples. The results are shown in FIG. 12.

As visible by the band shift in FIG. 12, Tris had the strongest effect on fragmentation (see “GR—high Tris” compared to “GR—low Tris”) for rat kidney and lung FFPE tissue. In particular, higher Tris concentrations increased the average size of DNA fragments, while low Tris concentrations decreased the average size. Furthermore, spermidine addition impacted the DNA fragment size. Addition of spermidine to a low Tris lysis composition increased the fragment size (see “Gr—low Tris+spermidine”) compared to the low Tris lysis composition (see “GR std”). As shown in FIG. 12, the degree of fragmentation was controlled by the addition of certain compounds (additives) to the system. Compounds showing an effect on fragmentation include Tris and spermidine. DTT, glycine, and spermine did not impact fragmentation in the present example in the same way. Tris had the strongest effect on fragmentation.

(b) The Incubation Time of UNG can be Reduced by Providing a Suitable Lysis Solution

The UNG activity was tested using a bisulfide DNA test. In bisulfide DNA the cytosine nucleobases of the DNA are exchanged with uracil nucleobases. The uracil nucleobases are cleaved by the UNG. As a result DNA is partially degraded, reducing the amount of recoverable DNA.

Workflow

The lysis compositions containing the bisulfide DNA were prepared as disclosed above with the exception that 2.5 μL of water were substituted with 2.5 μL of bis.gDNA having a concentration of 830 ng/μL. Then, the mixture was diluted with 115 μL water and 35 μL UNG were added. Samples were then incubated for 5 min at 50° C. Afterwards, the DNA was purified as explained in Example 1.1 (see point 6. Purification of DNA).

Control

As control a sample was incubated for 60 min at 50° C., as is performed in the GeneRead™ DNA FFPE workflow. As further controls no UNG digestion was performed (“w/o UNG”).

Results

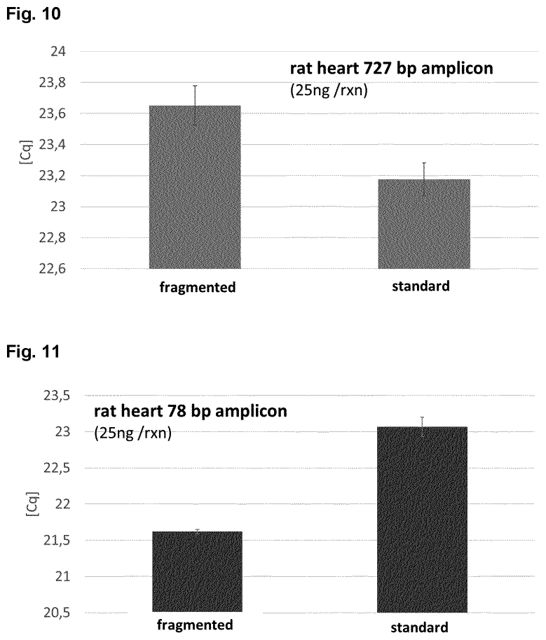

The results of the UNG activity test are shown in FIG. 13. In particular, 5 ng (dark shaded columns) or 10 ng (light shaded columns) extracted DNA was analyzed by quantitative Real Time PCR using an amplicon size of 110 bp and wobble bases in the primer sequence in order to ensure primer annealing. As before, the Ct values were determined. As the UNG digests the bisulfide DNA, it is desired to have a lower amount of recovered DNA and thus obtain higher Ct values.

As shown in FIG. 13, overall, good results were obtained for the UNG digestion except for the samples with higher Tris concentration and additional glycine, as well as for samples with a low Tris concentration and additional DTT. These have comparably a slightly lower Ct value. However, these still have significantly higher Ct values than the controls without UNG digestion. Importantly, the lysis solution samples were only incubated for 5 min compared to the control sample (“GR FFPE std”), which was incubated for 60 min. Yet, comparable Ct values were measured, demonstrating a similar UNG activity. Therefore, by providing a lysis composition according to the present disclosure, the UNG digestion time can be successfully reduced down to 5 min. A significant reduction in processing time is achieved.

(c) Conclusions

By starting with a diluted low salt lysis composition of Example 3, as described above, and optionally adding an additive such as Tris or spermidine, the extraction protocol can be optimized for either short or long fragments used in the downstream PCR. Moreover, processing time can be reduced by providing a lysis composition of the present disclosure, as the UNG digestion time is reduced from 60 min down to 5 min without a loss in activity.

5. Overall Conclusions

As demonstrated in the examples, the additional proteinase digestion performed after the de-crosslinking step significantly enhances the performance of the DNA purification from fixed biological samples, such as in particular FFPE samples. The results indicate that despite a first proteinase digestion step and a crosslink removal step, the nucleic acids such as DNA appear to still contain protein cross-links or similar protein related modifications of the DNA, which hamper downstream processes (e.g. PCR amplification or NGS). By performing the additional proteinase digestion step after de-crosslinking, these are removed, which improves the quality of the DNA, allowing for enhanced downstream processing and analysis. The improvements were verified for various reference protocols demonstrating that the method of the present disclosure leads to significant improvements over prior art methods. Moreover, by comparing performance of an extended first proteinase digestion (overnight) to the method of the present disclosure using the second proteinase digestion step after de-crosslinking, it was shown that not the overall digestion time is important but the particular sequence of steps. By performing a first proteinase digestion step, followed by a heating step to remove crosslinks, followed by an additional/second proteinase digestion step important improvements are achieved. This sequence of steps allows obtaining high quality nucleic acids (such as DNA), while allowing completing the workflow within a short time period. Thus, the method is very fast and at the same time highly effective in obtaining a high quality DNA.

The improvements in DNA yield and accessibility are also reflected in the improved NGS performance. Moreover, the method of the present disclosure with the tunable size modifications works well in all variants. In addition, it was possible to reduce the UNG incubation time from 60 min to 5 min, which demonstrates that the processing time can be significantly reduced. In all cases, the method according to the present invention shows a benefit compared to prior art methods, including the legacy QIAamp® FFPE kit. The method according to the present disclosure is a much faster and more convenient workflow in comparison, and still provides better UMI values.

### 6. Example 5: DNA Isolation from Different FFPE Tissue Samples Using the EZ1 Advanced XL Instrument

DNA was isolated from about 2 mm³ of different FFPE tissues, respectively, using the EZ1 Advanced XL instrument for bind wash/elute/steps. The EZ1 Advanced XL instrument is a robotic workstation for automated purification of nucleic acids.

Deparaffinization, lysis and cross-link removal was carried out as follows prior to processing the sample with the robotic instrument:
300 μl Deparaffinization Solution (DPS), vortex, spin
3 min at 56° C.
cool to room temperature
Mastermix: 55 μl RNase-free water, 25 μl Buffer FTB (QIAGEN) and 20 μl proteinase K per sample
vortex and spin sample
1 h at 56° C., 1000 rpm
1 h at 90° C.
remove upper layer
115 μl Rnase-free water
35 μl Rnase-free water
5 min at 50° C.
spin
2 μl RNaseA, 2 min at RT

Performance of this protocol was compared to a modified protocol according to the present invention that includes a second Proteinase K step prior to binding:
20 μl PK, vortex, 15 min at 65° C. 450 rpm

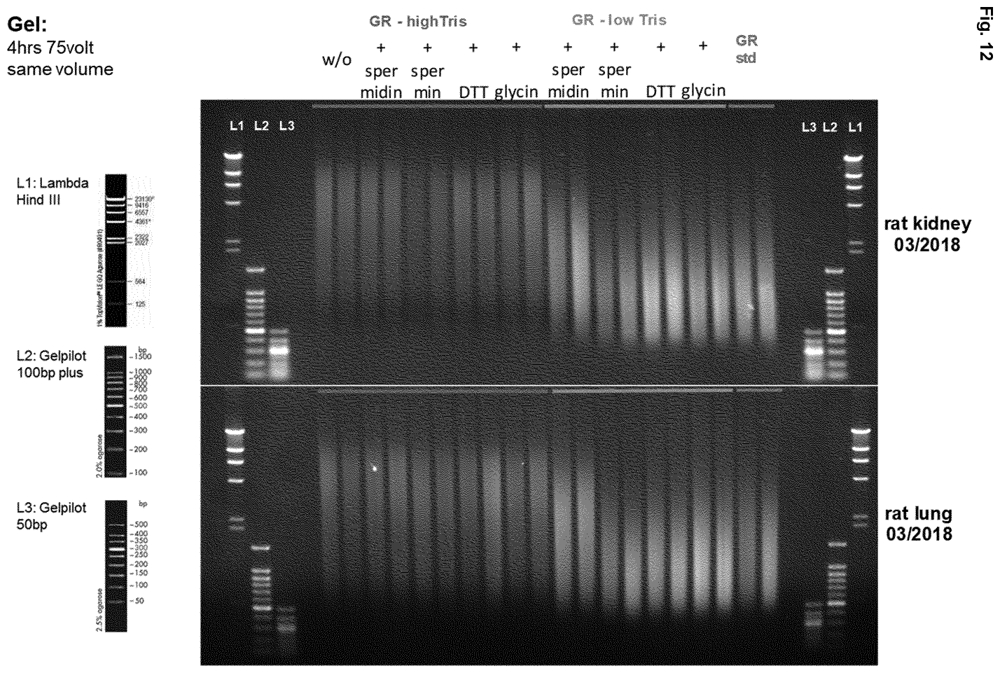

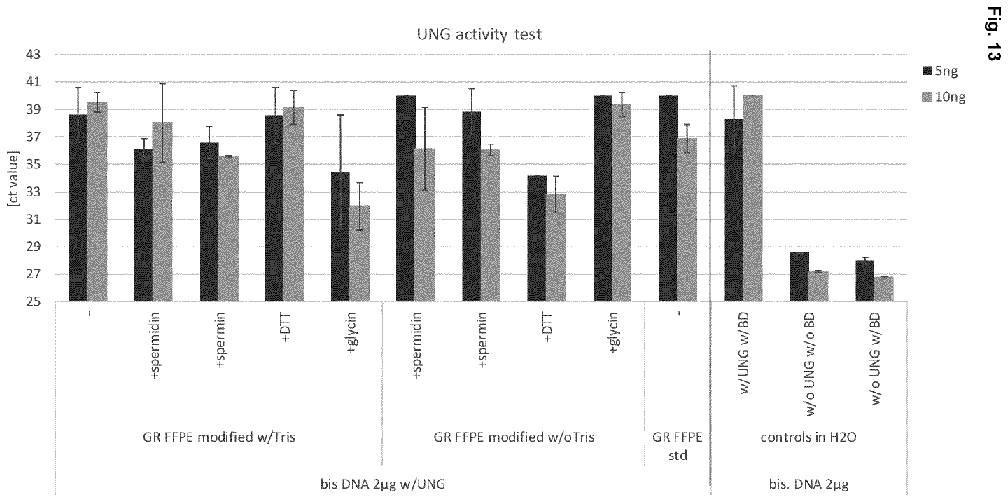

The yield was determined by UV VIS and Qubit dsDNA BR measurement for a human kidney and a human breast sample. FIG. 14 shows the results. The performance of the isolated DNA in qPCR was determined by adding the same amount of volume adjusted to the actual elution volume to each reaction. A 66 bp and a 500 bp of the human 18S rRNA gene was amplified from the eluates obtained after extraction with either protocol option. FIG. 15 shows the results.

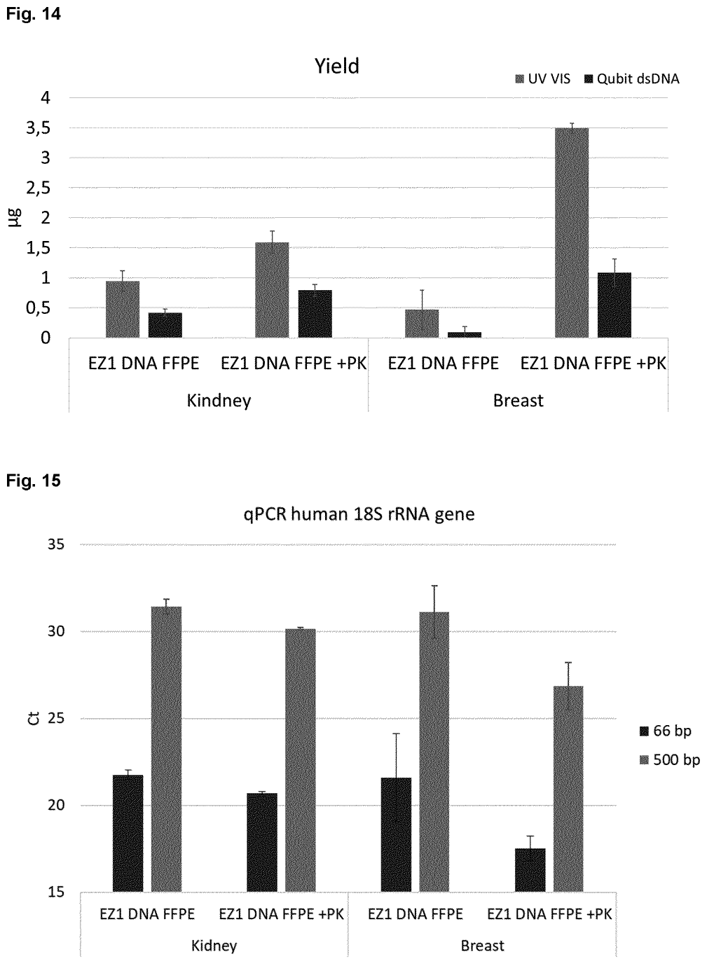

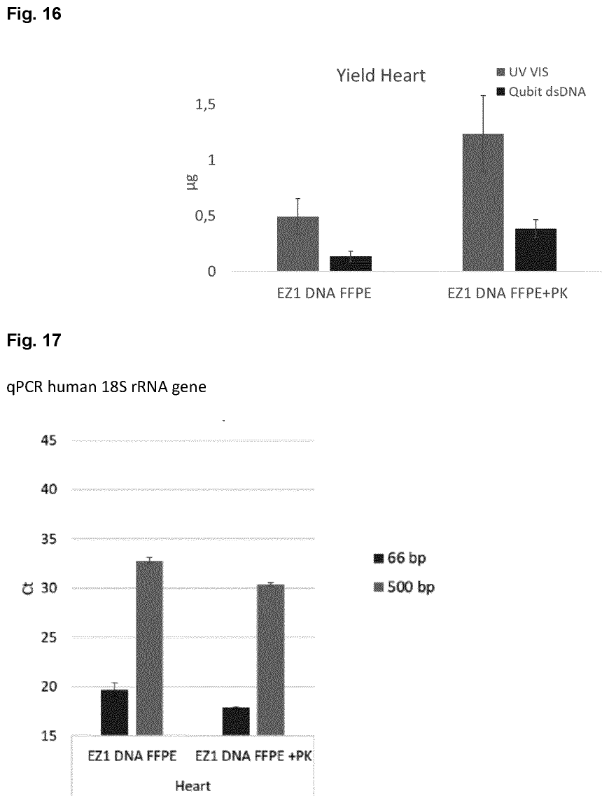

The experiment was repeated with about 2 mm³ of a human heart sample. The yield was again determined by UV VIS and Qubit dsDNA BR measurement and FIG. 16 shows the results. The performance of the isolated DNA in qPCR was again determined by adding the same amount of volume adjusted to the actual elution volume to each reaction. A 66 bp and a 500 bp of the human 18S rRNA gene was amplified from the eluates obtained after extraction with either protocol option and FIG. 17 shows the results.

Example 5 demonstrates based on UV VIS and Qubit dsDNA measurement as well as by qPCR that the inclusion of a 2^{nd} proteolytic digest according to the teachings of the present invention leads to an improvement in nucleic acid yield and thereby improves the performance in the qPCR.

1. A method for lysing a fixed biological sample, wherein the fixed biological sample comprises crosslinks between nucleic acid molecules and protein molecules due to the fixation, the method comprising
(a) lysing the fixed biological sample, wherein lysis involves digestion with a proteolytic enzyme;
(b) heating the lysed sample to reverse crosslinks;
(c) adding a proteolytic enzyme and performing a proteolytic digestion;
optionally wherein one or more additional treatment steps are performed between step (b) and step (c).

2. A method for obtaining purified nucleic acids from a fixed biological sample, comprising lysing the fixed biological sample according to the lysis method of claim 1, wherein subsequent to step (c) the method comprises
(d) purifying nucleic acids from the lysed sample.

3. The method according to claim 1 or 2, wherein lysis step (a) comprises preparing a lysis mixture, wherein the lysis mixture comprises
(i) the fixed biological sample, and
(ii) a lysis composition comprising the proteolytic enzyme.

4. The method according to claim 3, wherein step (a) has one or more of the following characteristics:
(i) the proteolytic enzyme is a protease, optionally a serine protease such as proteinase K;
(ii) the lysis mixture is heated to assist the digestion by the proteolytic enzyme, optionally wherein heating is performed at a temperature in the range of 35-75° C., optionally 40-70° C.;
(iii) the lysis mixture is incubated for at least 30 min, such as at least 45 min or at least 50 min, optionally wherein incubation occurs at an elevated temperature between 35-75° C. and/or wherein the sample is agitated during incubation; and/or
(iv) step (a) is completed in 120 min or less, optionally 100 min or less, 90 min or less or 70 min or less.

5. The method according to one or more of claims 1 to 4, wherein step (b) has one or more of the following characteristics:
(i) step (b) comprises heating the lysed sample to a temperature of at least 80° C., optionally at least 85° C. or at least 90° C.;
(ii) the lysed sample is heated to reverse crosslinks for at least 30 min, at least 45 min or at least 50 min; and/or
(iii) the lysed sample is heated at a temperature in a range of 80-110° C., such as 85-100° C. for 30-120 min, such as 45-90 min or 50-70 min.

6. The method according to one or more of claims 1 to 5, wherein step (c) has one or more of the following characteristics:
(i) the proteolytic enzyme is a protease, optionally a serine protease such as proteinase K;
(ii) step (c) comprises heating to assist the digestion by the proteolytic enzyme, optionally wherein heating is performed at a temperature in the range of 35-75° C., such as 40-70° C. or 50-70° C.;
(iii) step (c) comprises incubation for at least 5 min, such as at least 10 min or at least 15 min, optionally wherein incubation occurs at an elevated temperature between 35-75° C. and/or wherein the sample is agitated during incubation;
(iv) incubation in step (c) is shorter than incubation in step (a), and/or wherein the incubation temperature in step (c) is higher than in step (a); and/or
(v) step (c) is completed in 30 min or less, optionally 20 min or less.

7. The method according to one or more of claims 1 to 6, comprising performing at least one enzymatic treatment step different from a proteolytic digestion step between step (b) and step (c).

8. The method according to claim 7, wherein the at least one enzymatic treatment step involves the use of one or more of a glycosylase, a nuclease, a lipase or a combination of the foregoing.

9. The method according to claim 7 or 8, wherein the at least one enzymatic treatment step involves the use of a DNA glycosylase, such as a uracil DNA glycosylase, preferably a uracil-N-glycosylase.

10. The method according to claim 9, wherein glycosylase treatment step is performed at elevated temperature and/or wherein the glycosylase treatment step is completed in 30 min or less, 20 min or less, 15 min or less or 10 min or less, optionally wherein the glycosylase is a uracil-N-glycosylase.

11. The method according to one or more of claims 7 to 10, comprising diluting the sample obtained from step (b) for performing the at least one enzymatic treatment step prior to step (c).

12. The method according to any one of claims 7 to 11, wherein during the enzymatic treatment step the salt concentration in the enzymatic treatment mixture has one or more of the following characteristics:
(i) the salt concentration is ≤500 mM, and optionally is selected from ≤300 mM, ≤250 mM, ≤200 mM, ≤150 mM and ≤100 mM;
(ii) the salt concentration is within a range of 10 mM to 500 mM, such as within a range of 15 mM to 300 mM, 15 mM to 200 mM or 15 to 150 mM,
optionally wherein the at least one enzymatic treatment step involves the use of a DNA glycosylase, such as a uracil DNA glycosylase.

13. The method according to one or more of claims 3 to 12, wherein the lysis composition in step (a) has a pH in the range of 6.0 to 9.5, preferably 6.5 to 9.0 or 7.0 to 9.0.

14. The method according to one or more of claims 3 to 13, wherein the lysis composition in step (a) further comprises one or more, preferably all, of the following compounds:
(i) a salt;
(ii) a detergent;
(iii) a buffering agent.

15. The method according to one or more of claims 1 to 14, wherein the lysis composition comprises a reactive compound which has one or more of the following characteristics:
(i) the reactive compound reacts with the fixative or a chemical moiety released in heating step (b) and/or with a crosslink induced by the fixative, optionally wherein the fixative is an aldehyde-containing fixative, such as formaldehyde;
(ii) the reactive compound comprises a nucleophilic group, preferably an amine group;
(iii) the reactive compound comprises
one or more primary amine groups, optionally one primary amine group and one or more hydroxyl groups, preferably three hydroxyl groups; or
two primary amine groups and optionally one secondary amine group; and/or
(iv) the reactive compound is 2-amino-2-(hydroxymethyl)propane-1,3-diol or a derivative thereof or spermidine or a derivative thereof or a combination thereof.

16. The method according to claim 14 or 15, having one or more of the following characteristics;
(i) the reactive compound is present in the lysis composition and/or the lysis mixture in a concentration in a range of 1 mM to 500 mM, such as 5 mM to 500 mM;
(ii) the concentration of the reactive compound, preferably selected from Tris or spermidine, is chosen to control the length of the released nucleic acid molecules, wherein a lower concentration of the reactive compound in the lysis composition and/or the lysis mixture results in a higher degree of fragmentation of the released nucleic acid molecules.

17. The method according to one or more of claims 14 to 16, wherein the lysis composition has one or more of the following characteristics:
(a) the salt has one or more of the following characteristics:
(i) the salt is a mono- or divalent salt,
(ii) the salt is a chaotropic or non-chaotropic salt,
(iii) the salt is a non-buffering salt,
(iv) the salt is an alkali metal salt, optionally an alkali metal halide, and/or
(v) the salt is a chloride salt, optionally selected from sodium chloride, potassium chloride, lithium chloride and cesium chloride, wherein preferably the salt is sodium chloride;
(b) the detergent has one or more of the following characteristics:
(i) the detergent is a ionic or non-ionic detergent,
(ii) the detergent is an anionic detergent, and/or
(iii) the detergent is a sulfate or sulfonate of a fatty alcohol, such as sodium dodecyl sulfate, sodium dodecyl sulfonate or dodecylbenzenesulfonic acid, preferably the detergent is sodium dodecyl sulfate;
(c) it comprises a reactive compound according to claim 15 which is a buffering agent or the lysis composition comprises a buffering agent, optionally wherein the buffering agent has a pKa value which is in the range of 5.0 to 10.5, optionally the pKa value is selected from 5.5 to 10.0, 6.0 to 10.0, 6.5 to 10.0, 7.0 to 9.8 or 7.2 to 9.8, optionally wherein the buffering agent is Tris;
and/or
(d) optionally, the lysis composition comprises a chelating agent, wherein preferably the chelating agent is an aminopolycarboxylic acid, more preferably ethylenedinitrilotetraacetic acid (EDTA).

18. The method according to claim 17, wherein in the lysis composition and/or the lysis mixture
(a) the salt is present in a concentration of at least 15 mM, at least 50 mM, at least 75 mM or at least 100 mM, optionally wherein the salt concentration is in the range of 15 mM-500 mM, such as 50 mM to 350 mM, 75 mM to 300 mM, 100 mM to 250 mM or 125 mM to 200 mM; and/or
(b) the detergent is present in the lysis composition and/or the lysis mixture in a concentration of at least 0.01%, at least 0.02%, at least 0.03%, or at least 0.04%, optionally wherein the detergent concentration is in the range of 0.01-3.0%, such as 0.02-2.75%, 0.03 to 2.5% or 0.04 to 2.0%.

19. The method according to one or more of claims 3 to 18, wherein the lysis composition is prepared by combining a lysis solution with the proteolytic enzyme, optionally wherein preparing the lysis composition further comprises adding water or dilution buffer.

20. The method according to claim 19, having one or more of the following characteristics:
(i) the lysis solution comprises a reactive compound, optionally wherein
the reactive compound is a reactive compound as defined in claim 15, and/or
the reactive compound is present in the lysis solution in a concentration of at least 5 mM, in particular at least 10 mM, at least 20 mM or at least 30 mM;
(ii) the lysis solution comprises a reactive compound that is a buffering agent or the lysis solution further comprises a buffering agent;
(iii) the lysis solution comprises a reactive compound as defined in claim 15, optionally wherein
the reactive compound comprises one or more primary amine groups, preferably one primary amine group, and one, two or three hydroxyl groups, preferably three hydroxyl groups, optionally wherein the reactive compound is present in the lysis solution in a concentration of at least 10 mM, such as at least 20 mM, at least 40 mM, at least 60 mM or at least 75 mM; or
the reactive compound comprises two primary amine groups and preferably one secondary amine group, optionally wherein the reactive compound is present in the lysis solution in a concentration of at least 0.25 mM in the lysis solution, in particular at least 0.5 mM, at least 1 mM, at least 1.25 mM or preferably at least 1.5 mM;
(iv) the lysis solution comprises a salt, optionally wherein
the salt is a salt as defined in claim 17 a);
the salt is present is present in the lysis solution in a concentration of at least 50 mM, optionally at least 75 mM, at least 100 mM, at least 150 mM, at least 200 mM or at least 250 mM;
(v) the lysis solution comprises a detergent, optionally wherein
the detergent is a detergent as defined in claim 17 b);
the detergent is a ionic or non-ionic detergent, preferably an anionic detergent;
the detergent is present in the lysis solution in a concentration of at least 0.01% at least 0.01%, at least 0.02%, at least 0.03%, or at least 0.04%, optionally wherein the detergent concentration is in the range of 0.01-3.0%, such as 0.02-2.75%, 0.03 to 2.5% or 0.04 to 2.0%.

21. The method according to one or more of claims 1 to 20, having one or more of the following characteristics:
(i) the nucleic acids comprise or substantially consist of DNA;
(ii) the fixed biological sample is a cell-containing sample;
(iii) the fixed biological sample is a solid fixed biological sample, in particular a fixed tissue sample;
(iv) the fixed biological sample is a liquid fixed biological sample;
(v) the fixed biological sample is a sample that was fixed using a cross-linking fixative, optionally an aldehyde containing fixative, such as formaldehyde and/or paraformaldehyde; and/or
(vi) the fixed biological sample is an FFPE sample.

22. The method according to one or more of claims 2 to 21, wherein step (d) comprises binding nucleic acids to a solid phase; optionally washing the nucleic acid bound to the solid phase; and eluting the nucleic acid from the solid phase;
and/or
wherein the method further comprises (e) analyzing the purified nucleic acids, wherein analyzing optionally includes one or more of the following:
(i) amplifying the nucleic acids, preferably using a polymerase enzyme;
(ii) amplifying the nucleic acids using a large amplicon PCR and/or a short amplicon PCR, optionally wherein the large amplicon PCR is for nucleic acid molecules having a size of at least 500 nt and the short amplicon PCR is for nucleic acid molecules having a size of less than 500 nt, preferably 300 nt, 200 nt or 150 nt; and/or
(iii) performing a next generation sequencing method, said method optionally comprising
(aa) attaching a unique molecular identifier sequence to the nucleic acids, wherein each nucleic acid molecule comprises a different unique molecular identifier sequence;
(bb) amplifying the nucleic acids with the attached unique molecular identifier sequence; and
(cc) sequencing the nucleic acids.

23. The method according to one or more of claims 1 to 22, wherein the method comprises
(a) lysing the fixed biological sample, wherein lysis involves digestion with a proteolytic enzyme, wherein lysis step (a) comprises preparing a lysis mixture, wherein the lysis mixture comprises
(i) the fixed biological sample, and
(ii) a lysis composition comprising the proteolytic enzyme and wherein the lysis mixture is incubated for at least 30 min at an elevated temperature between 35-75° C., optionally wherein step (a) is completed in 120 min or less;
(b) heating the lysed sample to reverse crosslinks, wherein the lysed sample is heated at a temperature in a range of 80-110° C., such as 85-100° C., for 30-120 min, such as 45-90 min or 50-70 min;
(c) adding a proteolytic enzyme and performing a proteolytic digestion, wherein step (c) comprises incubation for at least 5 min, such as at least 10 min or at least 15 min, at an elevated temperature between 35-75° C., such as 40-70° C. or 50-70° C.,
wherein incubation in step (c) is shorter than incubation in step (a) and/or wherein the incubation temperature in step (c) is higher than in step (a).

24. The method according to claim 23, wherein the fixed biological sample is a solid fixed biological sample, optionally a FFPE sample, and wherein the method comprises performing at least one enzymatic treatment step different from a proteolytic digestion step between step (b) and step (c), wherein the at least one enzymatic treatment step involves the use of one or more of a glycosylase, a nuclease, a lipase or a combination of the foregoing.

25. The method according to claim 24, wherein the at least one enzymatic treatment step involves the use of a DNA glycosylase, such as a uracil DNA glycosylase, preferably a uracil-N-glycosylase, wherein the DNA glycosylase treatment step is performed at elevated temperature and completed in 30 min or less, 20 min or less, 15 min or less or 10 min or less, optionally wherein the method comprises diluting the sample obtained from step (b) for performing the DNA glycosylase treatment step prior to step (c).

26. The method according to claim 24 or 25, wherein at least a nuclease treatment step is performed in-between step (b) and step (c), wherein the nuclease is a ribonuclease, such as ribonuclease A.

27. The method according to any one of claims 24 to 26, wherein the enzymatic treatment step different from a proteolytic digestion step performed in-between step (b) and step (c) comprises:
adding a DNA glycosylase, more preferably a uracil DNA glycosylase; and
adding a ribonuclease, such as ribonuclease A.

28. The method according to one or more of claims 23 to 27, wherein
step (a) is characterized in that the proteolytic enzyme is a protease, such as proteinase K, step (a) is completed in 120 min or less, optionally 100 min or less, 90 min or less or 70 min or less and heating is performed at a temperature in the range of 35-75° C., optionally 40-70° C.; and
step (c) is characterized in that the proteolytic enzyme is a protease, such as proteinase K, step (c) is completed in 30 min or less, optionally 20 min or less and heating is performed at a temperature in the range of 35-75° C., optionally 40-70° C. or 50-70°; and
wherein incubation in step (c) is shorter than incubation in step (a) and wherein the incubation temperature in step (c) is higher than in step (a).

29. The method according to claim 28, wherein the sample is agitated during incubation in step (a) and step (c).

30. The method according to one or more of claims 2 to 29, comprising lysing the fixed biological sample according to the lysis method as defined in one or more of claims 23 to 29, preferably as defined in one or more of claims 26 to 29,
wherein subsequent to step (c) the method comprises
(d) purifying DNA from the lysed sample, wherein step (d) comprises
binding DNA to a solid phase;
optionally washing the DNA bound to the solid phase; and
eluting the DNA from the solid phase;
and wherein the method further comprises
(e) analyzing the purified DNA, wherein analyzing includes one or more of the following:
(i) amplifying the DNA, preferably using a polymerase enzyme;
(ii) amplifying the DNA using a large amplicon PCR and/or a short amplicon PCR, optionally wherein the large amplicon PCR is for DNA molecules having a size of at least 500 nt and the short amplicon PCR is for DNA molecules having a size of less than 500 nt, preferably 300 nt, 200 nt or 150 nt; and/or
(iii) performing a next generation sequencing method.

31. The method according to claim 30, wherein step (e) comprises performing a next generation sequencing method, said sequencing method comprising
(aa) attaching a unique molecular identifier sequence to the DNA molecules, wherein each DNA molecule comprises a different unique molecular identifier sequence;
(bb) amplifying the DNA molecules with the attached unique molecular identifier sequence; and
(cc) sequencing the DNA molecules.
